SEQUENCE LISTING (11 sequences)

SEQ ID NO: 1 - DNA, 3595 bp, Artificial
  Synthetic DNA sequence including optimized codon sequence coding for Nocardia NRRL5646 CAR peptide.
acaatctagaggccagcctggccataaggagatatacatatggccgttgactcccctgacgaacgtcttcagcgtcgtattgcacaactgtttgccgaggatgaacaagtcaaagccgcacgtcctcttgaggcagtctccgccgctgtctccgcaccaggtatgcgtcttgcccaaatcgcagcaactgtcatggccggttatgcagaccgtcctgctgccggtcaacgtgcattcgaacttaatactgacgacgctacgggccgtacttctcttcgtttgttgcctcgtttcgagactatcacctaccgtgagctgtggcagcgtgtgggtgaagttgctgccgcatggcatcatgacccggagaatcctctgcgtgcgggcgacttcgttgcgcttttgggctttaccagcattgattacgctaccctggaccttgccgatattcacttgggagcggtgacagtcccccttcaagcatctgccgccgtcagccaattgatcgctattttgacggagaccagcccgcgtcttctggccagcacaccagaacatctggatgccgcggtcgaatgtcttctggccggtacaacccctgagcgtttggttgtttttgactaccatccagaggatgacgaccagcgtgccgctttcgagtccgcgcgtcgtcgtttggccgacgccggttccctggtcatcgttgagacccttgatgccgtgcgtgctcgtggccgtgacttgcctgccgctccattgtttgtgcccgacacggatgatgatcctttggcgctgcttatttatactagcggtagcaccggtacacctaagggtgcgatgtacaccaaccgtcttgcggccacgatgtggcagggaaattccatgttgcagggaaactctcaacgtgtcggtattaacttgaactacatgccaatgagccatattgccggtcgtatttccctgttcggtgtgcttgcccgtggaggcactgcttactttgccgcgaaatccgatatgtccacgttgtttgaggacattggcttggttcgtccgacggaaattttctttgtccctcgtgtctgtgatatggttttccagcgttatcagtctgagctggatcgtcgttctgtcgccggtgcagatttggatacgctggaccgtgaagtgaaggcggacttgcgtcagaactacctgggtggtcgtttcttggttgccgtggtgggcagcgctccccttgccgcagaaatgaagacctttatggagtctgtccttgatctgcccttgcacgacggttacggttccacggaagcaggcgcttccgttctgctggataaccaaattcaacgtccacccgtgctggattacaagctggtggatgttcctgagctgggatactttcgtactgatcgtcctcacccacgtggtgaactgttgctgaaggccgaaacgacgattcctggctactacaagcgtcctgaggtcaccgcggaaattttcgatgaggacggtttttataaaacaggcgacattgtcgccgaattggagcatgatcgtcttgtctatgttgaccgtcgtaacaacgtcttgaagttgtcccagggtgagttcgttacagttgcgcacttggaagcagtctttgcctcctctcctcttatccgtcaaattttcatctacggctcctctgagcgttcctatcttcttgctgttattgtcccaactgacgatgcgctgcgtggacgtgacaccgccacgctgaaatccgcactggcggaatccatccaacgtatcgcaaaggacgccaatctgcaaccctacgaaattccccgtgattttctgatcgaaaccgagccgtttacaattgctaacggtcttttgtctggtattgccaaactgcttcgtcccaacctgaaggagcgttacggcgctcaacttgagcaaatgtacacggacctggccacaggccaagccgatgagttgcttgccctgcgtcgtgaagccgctgatttgcctgtgcttgaaacagtgagccgtgcggccaaagccatgttgggtgtcgcatctgcggacatgcgtcctgacgcccacttcaccgatttgggcggtgactcccttagcgccttgtccttttccaatcttcttcatgaaatttttggcgttgaagttccggtcggagtggtcgtgagcccggcaaacgagttgcgtgacttggctaactatattgaagccgaacgtaactctggtgccaagcgtcctacttttacttctgttcacggtggaggttccgaaattcgtgccgccgatttgactttggacaagtttatcgacgcccgtacgttggccgcagccgacagcatcccccatgctccagtcccggcccaaaccgttcttctgaccggtgccaacggttacctgggtcgtttcctgtgtcttgagtggcttgagcgtctggataaaaccggcggaacactgatttgcgttgttcgtggttccgacgccgctgccgcccgtaaacgtttggatagcgcgttcgactccggtgatcctggtcttctggagcactaccaacagttggctgcccgtacgctggaggttttggctggagacatcggtgatccgaatttgggccttgatgatgctacgtggcaacgtctggcggaaacggtggatttgatcgttcatcctgccgctttggtcaaccatgttttgccatatacccagctgtttggcccaaacgttgttggtactgctgaaatcgttcgtcttgcgattaccgctcgtcgtaagccagtcacctacttgtccaccgtgggtgtcgcggaccaagttgatcccgccgagtaccaagaggattccgatgttcgtgagatgtccgctgttcgtgttgtccgtgagtcctatgccaacggttacggaaactctaaatgggccggtgaggtgctgctgcgtgaagcacacgatctttgtggtctgccagttgcggtgttccgttccgacatgatcttggcccattctcgttatgctggccagcttaatgtccaagatgttttcactcgtctgatcctgtccctggtggctactggtatcgccccttattccttctatcgtactgacgctgatggtaaccgtcagcgtgcacattacgatggtttgccagctgacttcacggcggctgctatcaccgctttgggtattcaggccacagaaggattccgtacgtacgatgtgttgaatccttacgatgatggtatcagcctggacgagttcgtggactggcttgtggaatccggacatccgatccaacgtatcacggattactccgattggtttcatcgtttcgagactgcgattcgtgcacttcctgaaaagcaacgtcaggcctccgtgttgccgcttttggacgcttaccgtaacccttgcccagcggtgcgtggcgccattctgcctgccaaagagtttcaggctgcggttcaaaccgcgaagattggcccagaacaggacattcctcacctttctgctcccctgattgacaagtacgtgtctgatctggagcttttgcaattgttgtaatgaggccaaactggccaccatcaccatcacc

SEQ ID NO: 2 - protein, 1174 aa, Artificial
  Synthetic peptide sequence from optimized codon sequence coding for Nocardia NRRL5646 CAR peptide.
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 3 - DNA, 3525 bp, Artificial
  Synthetic DNA sequence including optimized codon sequence coding for Mycobacterium sp. (strain JLS) CAR peptide.
atgtccactgagacccgtgaagcccgtttgcagcaacgtattgctcacttgtttgccaccgacccccaatttgccgccgcccgtcccgaccctcgtatttctgacgccgttgatcgtgacgacgcacgtttgaccgccattgtgtctgctgtgatgtctggctatgcagatcgtcctgctcttggtcaacgtgcagcagagttcgctactgacccccagactggtcgtactacgatggaactgttgcctcgttttgacacgattacctaccgtgagttgctggatcgtgtgcgtgcccttaccaacgcctggcatgctgacggtgttcgtcctggagaccgtgttgcgattttgggctttaccggtattgattacactgttgttgaccttgccttgattcagttgggtgcagtcgcagtcccattgcaaaccagcgccgccgttgaagcccttcgtcccattgttgctgaaacagaacccatgttgattgccaccggagttgatcatgttgatgccgccgccgagcttgctcttacaggtcaccgtccgagccaggttgttgtgtttgaccatcgtgaacaagttgatgacgaacgtgacgctgtgcgtgccgctaccgcacgtttgggtgacgcagttccggtggagactttggcagaagtcttgcgtcgtggtgcccatctgcccgctgtcgcaccccacgtctttgacgaggccgatcctttgcgtttgctgatttacacctctggctctaccggcgctccgaagggtgcgatgtacccagagagcaaagtcgcaggcatgtggcgtgcaagcgcaaaggctgcctggaacaatgatcagacggcaattccgtctatcacgcttaacttcttgccgatgtctcacgtcatgggccgtggattgctgtgtggtactctttctactggcggtactgcgtattttgccgcacgttccgacctgtccacactgcttgaggacttgcgtcttgtccgtcccacccaattgtccttcgttccgcgtatttgggacatgctttttcaggagtttgtcggtgaagtcgatcgtcgtgttaacgacggtgcggaccgtcccactgctgaggctgatgtcttggccgagttgcgtcaggaactgttgggaggtcgttttgtcaccgcgatgaccggtagcgcccctatttcccctgagatgaaaacgtgggtggaaacgcttcttgacatgcaccttgttgaaggctatggttctacggaggctggcgctgtgttcgtggacggtcacattcaacgtcccccggttttggattacaaacttgttgacgttccagacctgggttactttagcacagaccgtccacatccgcgtggtgagctgcttgttcgttctacgcagttgtttccaggatactacaaacgtccagacgtgaccgccgaggttttcgatgatgatggcttctaccgtactggagatattgtggctgaattgggtcctgaccagttgcagtacctggaccgtcgtaataatgtcctgaaattggcgcagggcgagttcgtcactatcagcaaactggaagctgtgtttgccggttccgccctggtccgtcagatctttgtttatggcaactccgcccgttcttacttgttggccgtcgttgttcccactgatgacgccgttgcacgtcacgatcctgcctccctgaagacagctattagcgcttctttgcagcaggctgcgaaaacagcaggcttgcagagctatgaattgcctcgtgactttttggtggagacacaaccttttacactggaaaacggactgctgacgggtattcgtaagttggcacgtcctaagttgaaagcgcgttacggtgaccgtctggaggccttgtatgttgagcttgcagaaggccaggcaggtgaactgcgtactctgcgtcgtgatggtgcaaaacgtcctgtcgccgagacagttggccgtgccgccgccgccttgttgggtgcagctgcggcggatgtgcgtccagatgcccatttcaccgatcttggcggcgactctctgtccgccctgacttttggtaatttgttgcaggaaatcttcggtgttgacgttcccgtcggcgtcattgtctcccccgctgctgacttggcctccatcgctgcgtatattgaaacagagcaggcttccacgggtaaacgtccaacttatgcctccgttcatggtcgtgatgctgagcaagtccgtgcccgtgacttgacccttgataaattcatcgacgcagagacgttgtctgcggcgacagagttgccagtgccaatcggtgaagtgcgtaccgtgctgcttacaggcgctactggctttctgggtcgttacctggccctggattggctggaacgtatggcactggttgatggcaaagtgatctgcttggtccgtgcaaaagacgacgcagctgcgcgtaagcgtctggatgacaccttcgattccggtgaccctaaattgttggctcattaccgtaagttggccgctgatcacctggaggtcttggcgggcgacaagggcgaagccgatttgggtctgccacaccaggtgtggcaacgtttggctgacaccgtcgatcttatcgtggaccccgctgcgttggtcaatcatgtgctgccgtactctcaacttttcggacccaacgccctgggaacggcagagttgatccgtcttgccttgacgacccgtatcaaacctttcacctacgtttccaccattggtgttggcgcgggtattgagccgggtcgtttcacagaagacgacgacattcgtgttattagccctactcgtgccgttgatacgggttacgctaacggatatggtaactctaagtgggcaggtgaggttcttcttcgtgaggcccacgatctttgtggtctgccagttgcagtttttcgttgtgatatgattctggccgatacaacgtatgccggtcaactgaacctgccagatatgtttacccgtatgatggtctctttggtgacaaccggcattgccccgaagtcttttcatccacttgatgcgaagggccaccgtcagcgtgcccattatgacggtttgccagtggaatttgtcgctgaaagcatctctgccctgggtgcccaggctgtggacgaggctggcactggtttcgccacataccatgttatgaaccctcatgatgacggcattggccttgatgaatttgttgattggttggttgaagcgggttatcgtatcgaccgtattgacgactatgcagcctggcttcaacgttttgaaaccgctctgcgtgcactgcctgagcgtacacgtcagtactccttgctgccgttgcttcataattaccagcgtcccgctcatccaatcaacggtgctatggcccccacggaccgtttccgtgcggcagttcaggaggctaagttgggtcctgacaaagacattccgcatgttactcctggtgtcatcgttaagtacgccacagatttggaattgcttggcctgatttaa

SEQ ID NO: 4 - protein, 1174 aa, Artificial
  Synthetic peptide sequence from optimized codon sequence coding for Mycobacterium sp. (strain JLS) CAR peptide.
MSTETREARLQQRIAHLFATDPQFAAARPDPRISDAVDRDDARLTAIVSAVMSGYADRPALGQRAAEFATDPQTGRTTMELLPRFDTITYRELLDRVRALTNAWHADGVRPGDRVAILGFTGIDYTVVDLALIQLGAVAVPLQTSAAVEALRPIVAETEPMLIATGVDHVDAAAELALTGHRPSQVVVFDHREQVDDERDAVRAATARLGDAVPVETLAEVLRRGAHLPAVAPHVFDEADPLRLLIYTSGSTGAPKGAMYPESKVAGMWRASAKAAWNNDQTAIPSITLNFLPMSHVMGRGLLCGTLSTGGTAYFAARSDLSTLLEDLRLVRPTQLSFVPRIWDMLFQEFVGEVDRRVNDGADRPTAEADVLAELRQELLGGRFVTAMTGSAPISPEMKTWVETLLDMHLVEGYGSTEAGAVFVDGHIQRPPVLDYKLVDVPDLGYFSTDRPHPRGELLVRSTQLFPGYYKRPDVTAEVFDDDGFYRTGDIVAELGPDQLQYLDRRNNVLKLAQGEFVTISKLEAVFAGSALVRQIFVYGNSARSYLLAVVVPTDDAVARHDPASLKTAISASLQQAAKTAGLQSYELPRDFLVETQPFTLENGLLTGIRKLARPKLKARYGDRLEALYVELAEGQAGELRTLRRDGAKRPVAETVGRAAAALLGAAAADVRPDAHFTDLGGDSLSALTFGNLLQEIFGVDVPVGVIVSPAADLASIAAYIETEQASTGKRPTYASVHGRDAEQVRARDLTLDKFIDAETLSAATELPVPIGEVRTVLLTGATGFLGRYLALDWLERMALVDGKVICLVRAKDDAAARKRLDDTFDSGDPKLLAHYRKLAADHLEVLAGDKGEADLGLPHQVWQRLADTVDLIVDPAALVNHVLPYSQLFGPNALGTAELIRLALTTRIKPFTYVSTIGVGAGIEPGRFTEDDDIRVISPTRAVDTGYANGYGNSKWAGEVLLREAHDLCGLPVAVFRCDMILADTTYAGQLNLPDMFTRMMVSLVTTGIAPKSFHPLDAKGHRQRAHYDGLPVEFVAESISALGAQAVDEAGTGFATYHVMNPHDDGIGLDEFVDWLVEAGYRIDRIDDYAAWLQRFETALRALPERTRQYSLLPLLHNYQRPAHPINGAMAPTDRFRAAVQEAKLGPDKDIPHVTPGVIVKYATDLELLGLI

SEQ ID NO: 5 - DNA, 3517 bp, Artificial
  Synthetic DNA sequence including optimized codon sequence coding for Streptomyces griseus CAR peptide.
acaatctagaggccagcctggccataaggagatatacatatggctgaaccccttgatgccgcaaccgcctccgcacacgaccctggacaaggtttggcagaagcccttgccgccgtggaacctggtcgtgcccttgctgaagttatggcttccgttttggaaggtcacggtgatcgtccggctttgggcgagcgtgctcgtgaacccgaaactggacgtttgttgcctcattttgatacgatctcctatcgtgagctttggtctcgtgtgcgtgctttggccggtcgttggcatcatgaccctgagtaccctctgggtcccggagaccgtatctgcaccctgggctttaccagcacagattatgccaccctggatcttgcttgcatccacctgggtgctgttccagttccattgccatccaacgctccattgccccgtttggcgccggtggttgaggagtccggcccaaccgttcttgctgcatccgttgatcgtttggatactgcgattgatgttgtcctggccagctctaccatccgtcgtctgttggtcttcgatgatggacctggtgccacccgtccaggaggtgcccttgccgcagcccgtcaacgtctgtccggttccccggtcaccgtggacactctggccggtcttatcgaccgtggccgtgaccttccccccccacccctttatattcctgatcctggcgaggaccctctggctctgctgatttacacgtccggatctacaggcgcaccaaaaggcgcaatgtacactcaacgtctgctgggtacagcatggtacggtttcagctacggcgccgccgatacccctgccatttccgttctgtatctgccacagtcccacctggctggtcgttacgctgtcatgggtagcttggtgaaaggtggtactggatactttacggcagcggatgacttgtccacccttttcgaagacattgcgcttgtccgtcccaccgaattgacgatggttcctcgtctgtgtgacatgcttctgcaacactatcgttctgagcgtgatcgtcgtgcggacgagcccggtgatattgaagctgccgtcactaaagccgttcgtgaggacttcctgggtggacgtgtggcgaaggctttcgttggtactgcccctctttccgccgagttgacggctttcgtcgaatccgtgttgggttttcacctgtacactggctatggaagcacggaagccggtggtgtcttgttggatactgttgttcagcgtcctcccgtcacagactacaaactggtcgatgtgcctgagctgggatattatgctaccgatctgccgcatcctcgtggagagttgcttttgaaatcccacaccttgattcctggttattaccgtcgtcccgacctgaccgccgccatctttgacgccgacggttactaccgtaccggcgatgtttttgctgaaaccggtccggatcgtcttgtctatgttgaccgtactaaagacacgttgaagctgtctcagggtgagttcgttgccgtgtcccgtttggaaacagtcttgttggactctcctcttgttcaacacttgtacctttatggcaactctgagcgtgcatatttgcttgcggtcgtcgttcctacgccagatgcgttggctggttgtggaggcgacacggaagccctgcgtccgttgctgatggagtccctgcgttctgtcgcacgtcgtgccggtttgaacgcttacgaaatccctcgtggtatcttggtcgaaccggaaccttttagcccggagaacggtctgttcaccgagtctcataagttgctgcgtccacgtcttaaagaacgttatggtcctgctttggagttgctgtacgatcgtcttgccgacggtcaggatcgtcgtttgcgtgagcttcgtcgtactggtgccgaccgtcctgtccaggagaccgtgctgcgtgccgctcaagccttgttgggctccccaggctctgacttgcgtcccggcgctcactttacggatcttggtggcgactctttgtccgcagtcagcttttccgagttgatgaaggaaatttttcatgtcgatgttcctgttggcgccattattggtccagccgctgacctggccgaagttgcgcgttacatcactgctgctcgtcgtcctgcgggagcgccccgtccaacgccagcctccgttcatggcgaacatcgtactgaggtccgtgccggtgatctggccccagagaagtttttggatgcgccgactctggcagcagcccccgctttgccacgtccagacggtgacgttcgtactgtgttgctgaccggtgccactggctacttgggtcgttttctgtgtttggaatggctggagcgtttggcgccaagcggtggtcgtcttgtttgtttggttcgtggtagcgacgcaacggtcgcggcccgtcgtctggaggcggcgtttgactctggcgacacggcacttttgcgtcgttatcgtaaggccgcaggaaaaacgttggatgttgtcgccggtgacattggcgagcctttgctgggtctggccgaggagacctggcgtgagcttgctggtgccgtcgatttgattgtgcaccctgccgctcttgtcaaccacttgttgccctacggtgagctgttcggtccaaatgtcgtgggcaccgctgaggcgatccgtctggccctgaccacccgtttgaagcctgttaatcacgtttccaccgtggcggtgtgcctgggtacgcccgccgagaccgccgacgaaaacgctgatattcgtgccgctgttccggtgcgtacaacaggtcaaggctatgccgacggttacgcgacctctaaatgggctggcgaggtccttcttcgtgaggcacatgaacgttacggtttgccagtggctgtctttcgttctgacatggttttggcacaccgtacttacactggccaagttaacgtcccagatgttttgacacgtttgttgcttagccttgtggcgactggcatcgccccaggttctttttaccgtaccgatacacgtgcccactatgacggcctgccagtggactttaccgccgaggctgttgtggcactgggagcccccattactgagggtcaccgtacgttcaacgttctgaacccccacgatgacggtgtgagccttgatacttttgtggattggttgatcgaggcaggtcatcctattcgtcgtatcgacgatcatggtgcttggttgactcgtttcaccgccgccttgcgtgcgctgcctgagaagcaacgtcaacactccttgttgccgcttatcggtgcctgggcggagcccggcgaaggtgcccccggtccccttctgccagcacgtcgttttcatgcagcggtccgtgcagccggtgtgggtcctgaacgtgatattccgcgtgtctcccctgatttgattcgtaagtacgtgaccgatttgcgtgccctgggtttgttggcaggtccgtaatgaggccaaactggccaccatcaccatcacc

SEQ ID NO: 6 - protein, 1148 aa, Artificial
  Synthetic peptide sequence from optimized codon sequence coding for Streptomyces griseus CAR peptide.
MAEPLDAATASAHDPGQGLAEALAAVEPGRALAEVMASVLEGHGDRPALGERAREPETGRLLPHFDTISYRELWSRVRALAGRWHHDPEYPLGPGDRICTLGFTSTDYATLDLACIHLGAVPVPLPSNAPLPRLAPVVEESGPTVLAASVDRLDTAIDVVLASSTIRRLLVFDDGPGATRPGGALAAARQRLSGSPVTVDTLAGLIDRGRDLPPPPLYIPDPGEDPLALLIYTSGSTGAPKGAMYTQRLLGTAWYGFSYGAADTPAISVLYLPQSHLAGRYAVMGSLVKGGTGYFTAADDLSTLFEDIALVRPTELTMVPRLCDMLLQHYRSERDRRADEPGDIEAAVTKAVREDFLGGRVAKAFVGTAPLSAELTAFVESVLGFHLYTGYGSTEAGGVLLDTVVQRPPVTDYKLVDVPELGYYATDLPHPRGELLLKSHTLIPGYYRRPDLTAAIFDADGYYRTGDVFAETGPDRLVYVDRTKDTLKLSQGEFVAVSRLETVLLDSPLVQHLYLYGNSERAYLLAVVVPTPDALAGCGGDTEALRPLLMESLRSVARRAGLNAYEIPRGILVEPEPFSPENGLFTESHKLLRPRLKERYGPALELLYDRLADGQDRRLRELRRTGADRPVQETVLRAAQALLGSPGSDLRPGAHFTDLGGDSLSAVSFSELMKEIFHVDVPVGAIIGPAADLAEVARYITAARRPAGAPRPTPASVHGEHRTEVRAGDLAPEKFLDAPTLAAAPALPRPDGDVRTVLLTGATGYLGRFLCLEWLERLAPSGGRLVCLVRGSDATVAARRLEAAFDSGDTALLRRYRKAAGKTLDVVAGDIGEPLLGLAEETWRELAGAVDLIVHPAALVNHLLPYGELFGPNVVGTAEAIRLALTTRLKPVNHVSTVAVCLGTPAETADENADIRAAVPVRTTGQGYADGYATSKWAGEVLLREAHERYGLPVAVFRSDMVLAHRTYTGQVNVPDVLTRLLLSLVATGIAPGSFYRTDTRAHYDGLPVDFTAEAVVALGAPITEGHRTFNVLNPHDDGVSLDTFVDWLIEAGHPIRRIDDHGAWLTRFTAALRALPEKQRQHSLLPLIGAWAEPGEGAPGPLLPARRFHAAVRAAGVGPERDIPRVSPDLIRKYVTDLRALGLLAGP

SEQ ID NO: 7 - DNA, 739 bp, Artificial
  Synthetic DNA sequence including optimized codon sequence coding for Nocardia NRRL5646 PPTase peptide.
acaatctagaggccagcctggccataaggagatatacatatgatcgaaactattttgcccgcaggtgttgaatctgccgagcttttggaatatcctgaggaccttaaagcccaccctgctgaagaacatcttattgccaaatccgtcgaaaaacgtcgtcgtgatttcattggtgcccgtcattgcgcgcgtctggccctggccgagttgggcgagccacccgtcgcaattggcaaaggtgaacgtggtgcccctatttggccgcgtggcgttgtcggctctcttacccactgcgacggctaccgtgccgccgcagtcgcccataagatgcgtttccgttctattggcattgacgccgaaccgcacgccacccttcctgaaggagtcctggactctgtttctcttccacctgaacgtgagtggttgaagaccactgattctgctttgcatcttgatcgtttgttgttctgcgcgaaggaagcaacttataaggcttggtggccattgaccgctcgttggcttggctttgaggaagcacatattacttttgagattgaggatggtagcgccgatagcggcaatggtacttttcatagcgaactgttggttcctggtcagacgaatgacggtggtactcctcttcttagctttgatggacgttggctgattgccgatggttttatcttgaccgcaattgcgtatgcgtaatgaggccaaactggccaccatcaccatcacc

SEQ ID NO: 8 - protein, 222 aa, Artificial
  Synthetic peptide sequence from optimized codon sequence coding for Nocardia NRRL5646 PPTase peptide.
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 9 - protein, 1178 aa, Artificial
  Synthetic peptide having GDIH at N-terminal of CAR peptide from Nocardia NRRL5646.
GDIHMAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 10 - protein, 1152 aa, Artificial
  Synthetic peptide having GDIH at N-terminal of CAR peptide from Streptomyces griseus.
GDIHMAEPLDAATASAHDPGQGLAEALAAVEPGRALAEVMASVLEGHGDRPALGERAREPETGRLLPHFDTISYRELWSRVRALAGRWHHDPEYPLGPGDRICTLGFTSTDYATLDLACIHLGAVPVPLPSNAPLPRLAPVVEESGPTVLAASVDRLDTAIDVVLASSTIRRLLVFDDGPGATRPGGALAAARQRLSGSPVTVDTLAGLIDRGRDLPPPPLYIPDPGEDPLALLIYTSGSTGAPKGAMYTQRLLGTAWYGFSYGAADTPAISVLYLPQSHLAGRYAVMGSLVKGGTGYFTAADDLSTLFEDIALVRPTELTMVPRLCDMLLQHYRSERDRRADEPGDIEAAVTKAVREDFLGGRVAKAFVGTAPLSAELTAFVESVLGFHLYTGYGSTEAGGVLLDTVVQRPPVTDYKLVDVPELGYYATDLPHPRGELLLKSHTLIPGYYRRPDLTAAIFDADGYYRTGDVFAETGPDRLVYVDRTKDTLKLSQGEFVAVSRLETVLLDSPLVQHLYLYGNSERAYLLAVVVPTPDALAGCGGDTEALRPLLMESLRSVARRAGLNAYEIPRGILVEPEPFSPENGLFTESHKLLRPRLKERYGPALELLYDRLADGQDRRLRELRRTGADRPVQETVLRAAQALLGSPGSDLRPGAHFTDLGGDSLSAVSFSELMKEIFHVDVPVGAIIGPAADLAEVARYITAARRPAGAPRPTPASVHGEHRTEVRAGDLAPEKFLDAPTLAAAPALPRPDGDVRTVLLTGATGYLGRFLCLEWLERLAPSGGRLVCLVRGSDATVAARRLEAAFDSGDTALLRRYRKAAGKTLDVVAGDIGEPLLGLAEETWRELAGAVDLIVHPAALVNHLLPYGELFGPNVVGTAEAIRLALTTRLKPVNHVSTVAVCLGTPAETADENADIRAAVPVRTTGQGYADGYATSKWAGEVLLREAHERYGLPVAVFRSDMVLAHRTYTGQVNVPDVLTRLLLSLVATGIAPGSFYRTDTRAHYDGLPVDFTAEAVVALGAPITEGHRTFNVLNPHDDGVSLDTFVDWLIEAGHPIRRIDDHGAWLTRFTAALRALPEKQRQHSLLPLIGAWAEPGEGAPGPLLPARRFHAAVRAAGVGPERDIPRVSPDLIRKYVTDLRALGLLAGP

SEQ ID NO: 11 - protein, 226 aa, Artificial
  Synthetic peptide having GDIH at N-terminal of PPTase peptide from Nocardia NRRL5646.
GDIHMIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

(19) United States
(12) Patent Application Publication
(10) Pub. No.: US 2010/0298612 A1
(43) Pub. Date: Nov. 25, 2010

(54) ENGINEERED BIOSYNTHESIS OF FATTY ALCOHOLS

(75) Inventors: Behnaz Behrouzian, Sunnyvale, CA (US); Robert McDaniel, Palo Alto, CA (US); Xiyun Zhang, Fremont, CA (US); Louis Clark, San Francisco, CA (US)

Correspondence Address: Codexis, Inc., 200 Penobscot Drive, Redwood City, CA, 94063, US

(73) Assignee: CODEXIS, INC., Redwood City, CA (US)

(21) Appl. No.: 12/784,770

(22) Filed: May 21, 2010

Related U.S. Application Data
(60) Provisional application No. 61/180,534, filed on May 22, 2009.

(51) Int. Cl. C07C 31/125; C12P 7/02; C12N 1/21; C12N 1/15; C12N 1/19; C12N 1/13; C12P 7/24; C12N 9/02

(52) U.S. Cl. 568/840; 435/155; 435/252.3; 435/254.11; 435/254.2; 435/257.2; 435/147; 435/189

(57) ABSTRACT

The present disclosure provides a process for the production of long chain fatty alcohols by recombinant host cells expressing one or more heterologous carboxylic acid reductase enzymes useful for the conversion of fatty acids, and derivatives thereof, to long chain fatty alcohols.

### 1. CROSS-REFERENCE TO RELATED APPLICATIONS

This application claims benefit of provisional application No. 61/180,534 filed May 22, 2009, the entire content of which is incorporated herein by reference.

### 2. FIELD OF THE INVENTION

This invention relates to recombinant microorganisms which include polynucleotides encoding heterologous carboxylic acid reductases and the production of fatty alcohols having between C8 and C24 carbons in length as well as methods of their use.

### 3. BACKGROUND

The non-renewable nature and cost of fossil fuels have sparked interest in alternative energy sources including nuclear power, solar energy, wind power, as well as biological processes for production of fuels (“biofuels”). The latter biological approaches are particularly valuable in that they represent a renewable source of combustible materials which are not derived from petroleum sources. One option for producing biofuels includes the use of biomass to provide sugars for microbial (e.g., yeast) fermentations with the ultimate production of short chain alcohols such as ethanol and butanol. However, another alternative which includes the use of renewable carbon substrates includes the production of fatty acid derivatives such as fatty acid esters or fatty alcohols which may be used as a biofuel. The physical properties of fatty acids make them very suitable for fuel applications. Therefore fatty acid derived molecules such as fatty alcohols could be highly desirable products for biodiesel and/or jet fuel targets.

### 4. SUMMARY

The present disclosure has multiple aspects.

In one aspect, the invention relates to a recombinant microorganism comprising a nucleic acid sequence encoding a heterologous carboxylic acid reductase (CAR), wherein the recombinant microorganism is capable of producing fatty alcohols having C8 to C24 carbons in length. In one embodiment, the recombinant microorganism is capable of producing fatty alcohols having C10 to C20 carbons in length. In other embodiments, the carboxylic acid reductase is selected from the group consisting of a Mycobacterium carboxylic acid reductase, a Nocardia carboxylic acid reductase, and a Streptomyces griseus carboxylic acid reductase. In another embodiment, the carboxylic acid reductase has at least 90% sequence identity to SEQ ID NO: 2, at least 90% sequence identity to SEQ ID NO: 4, or at least 90% sequence identity to SEQ ID NO: 6. In some embodiments, the sequence identity is at least 95% to SEQ ID NO: 2, at least 95% to SEQ ID NO: 4, or at least 95% to SEQ ID NO: 6. In some embodiments, the sequence identity is at least 95% to SEQ ID NO: 2, at least 95% to SEQ ID NO: 4, or at least 95% to SEQ ID NO: 6. In other embodiments, the polynucleotide sequence encoding a CAR has at least 90% sequence identity to SEQ ID NO: 1, at least 90% sequence identity to SEQ ID NO: 3, or at least 90% sequence identity to SEQ ID NO: 5. In further embodiments, the recombinant microorganism is a bacterial strain, a filamentous fungal strain, a yeast strain or an algal strain. In other embodiments, the recombinant microorganism comprises a gene encoding a phosphopantetheinyl transferase polypeptide capable of attaching a pantetheine moiety to the carboxylic acid reductase.

In a second aspect, the invention relates to an isolated CAR variant, the variant comprising at least 90% sequence identity to SEQ ID NO: 4 and an amino acid substitution at one or more of the following positions 8270, A271, K274, A275, P467, Q584, E626, and/or D701 in SEQ ID NO: 4. In some embodiments, the variant is R270W, A271W, K274(G/N/V/I/W/L/M/Q/S), A275F, P467S, Q584, E626G, and/or D701G. In some embodiments, the variant comprises at least 90% sequence identity to SEQ ID NO: 4 and a combination of substitutions selected from K274L/A369T/L380Y, K274LN358H/E845A, K274M/T282K, K274Q/T282Y, K274S/A715T, K274W/L380G/A477T, K274W/T282E/L380V, K274W/T282Q, K274W/V358R and/or R43c/K274I when aligned with SEQ ID NO: 4.

In a third aspect, the invention relates to a process for the biologically-derived production of fatty alcohols comprising a) culturing a recombinant microorganism encompassed by the invention in an aqueous nutrient medium comprising an assimilable source of carbon under suitable culture conditions for a sufficient period of time to allow the production the fatty alcohols and b) recovering the fatty alcohols produced by the recombinant microorganism. In one embodiment, the culturing step is carried out a temperature within the range of from about 10° C. to about 80° C. In another embodiment, the culturing step is carried out for a period of from about 8 hours to about 240 hours. In a further embodiment, the amount of biologically produced fatty alcohol is in the range of 2 mg/L to 200 g/L of fermentation broth. In a further embodiment, the amount of biologically produced C14 to C18 fatty alcohols is in the range of 2 mg/L to 200 g/L. In yet other embodiments, the production of fatty alcohols having C10 to C20 carbons in length comprise at least 80% of the total isolated fatty alcohols. In another embodiment, the process further comprises reducing the fatty alcohols to corresponding alkanes.

In a fourth aspect, the invention relates to a method of catalytically reducing a fatty acid substrate to a corresponding C8 to C24 carbon containing fatty aldehyde comprising a) mixing an effective amount of a carboxylic acid reductase with a fatty acid substrate and co-substrates selected from ATP, NAD(H) and/or NADP(H) and b) incubating the mixture for a period of time and under conditions suitable to achieve reduction of the fatty acid substrate to the corresponding fatty aldehyde. In one embodiment, the fatty aldehyde is further reduced to a fatty alcohol.

In a fifth aspect, the invention relates to a process for the biologically-derived production of fatty alcohols in yeast which comprises culturing a recombinant yeast cell, which comprises a polynucleotide encoding a heterologous carboxylic acid reductase (CAR), said CAR comprising an amino acid sequence having at least 85% sequence identity (that is at least 85%, at least 88%, at least 90%, 91%, 92%, 93%, 94%, 95%, 96%, 97%, 98%, 99% and even 100% sequence identity) to SEQ ID NOs: 2, 4, 6, 9 or 10 under suitable culture conditions to allow expression of said CAR and production of the fatty alcohols and recovering the produced fatty alcohol. In some embodiments, the yeast is a Yarrowia strain or Saccharomyces strain. In some embodiments, the amount of fatty alcohol produced is at least 2.0 mg/L. In further embodiments, the yeast are capable of producing fatty alcohols comprising C10 to C20 carbons in length. In other embodiments, the recombinant yeast further comprises a gene encoding a phosphopantetheinyl transferase. The phosphopantetheinyl transferase (PPTase) may be a heterologous PPTase, such as but not limited to a PPTase derived from a Nocardia strain or Mycobacterium strain. In other embodiments, the recombinant yeast cells comprise a polynucleotide that encodes a heterologous alcohol dehydrogenase. In yet other embodiments of this aspect, the invention relates to a biologically-derived fatty alcohol composition comprising the fatty alcohols or derivative thereof produced according to the process.

In yet a further aspect, the invention relates to a process for the biologically-derived production of fatty alcohols comprising culturing a recombinant microorganism, which comprises a gene coding for a heterologous carboxylic acid reductase (CAR) comprising an amino acid sequence having at least 90% sequence identity to SEQ ID NOs: 2, 4, 6, 9 or 10 and a polynucleotide coding for a heterologous phosphopantetheinyl transferase (PPTase) having at least 80% sequence identity to SEQ ID NOs: 8 or 11, wherein said PPTase is capable of attaching a phosphopantetheine moiety to the CAR and culturing under suitable culture conditions to allow to expression of the CAR and PPTase and production of the fatty alcohols, and recovering the fatty alcohols produced by the recombinant microorganism. In some embodiments, the recombinant microorganism is a bacterial, yeast, filamentous fungal or algal strain. In other embodiments, the CAR and PPTase are derived from the same organism.

### 5. BRIEF DESCRIPTION OF THE FIGURES

FIG. 1 depicts the replicative Y. lipolytica vector pCEN351 (8789 bp) containing cassettes encoding phleomycin (Ble) and hygromycin (HygB) resistance. Ars68 is an autonomous replicating sequence isolated from Y. lipolytica chromosomal DNA.

FIG. 2 depicts the expression vector pCEN364 comprising the Mycobacterium sp JLS gene encoding carboxylic acid reductase (CAR).

FIGS. 3A and 3B illustrate the codon optimized polynucleotide sequence (SEQ ID NO: 1) encoding a CAR (SEQ ID NO: 2) of Nocardia NRRL5646. The 5′ and 3′ polynucleotide flanking regions are in italics and the first ATG coding for methionine in the expressed protein is underlined and in bold. Stop codons are identified by “*”. The flanking regions upstream and downstream of the stop codon are not expressed. Conceptual translation of the longest open reading frame (ORF) in SEQ ID NO: 1 resulted in SEQ ID NO: 9. The initiator methionine (underlined and in bold) of the CAR protein is residue 5 of SEQ ID NO: 9.

FIGS. 4A and 4B illustrate the codon optimized polynucleotide sequence (SEQ ID NO: 3) encoding a CAR (SEQ ID NO: 4) of Mycobacterium sp. (strain JLS).

FIGS. 5A and 5B illustrate the codon optimized polynucleotide sequence (SEQ ID NO: 5) encoding a CAR (SEQ ID NO: 6) of Streptomyces griseus. The 5′ and 3′ polynucleotide flanking regions are in italics and the first ATG coding for methionine in the expressed protein is underlined and in bold. Stop codons are identified by “*”. The flanking regions upstream and downstream of the stop codon are not expressed. Conceptual translation of the longest open reading frame (ORF) in SEQ ID NO: 5 resulted in SEQ ID NO: 10. The initiator methionine (underlined and in bold) of the CAR protein is residue 5 of SEQ ID NO: 10.

FIGS. 6a and 6B illustrate the codon optimized polynucleotide sequence (SEQ ID NO: 7) encoding a Nocardia NRRL 5646 phosphopantetheinyl transferase (PPTase) (SEQ ID NO: 8). The 5′ and 3′ polynucleotide flanking regions are in italics and the first ATG coding for methionine in the expressed protein is underlined and in bold. Stop codons are identified by “*”. The flanking regions upstream and downstream of the stop codon are not expressed. Conceptual translation of the longest open reading frame (ORF) in SEQ ID NO: 10 resulted in SEQ ID NO: 11. The initiator methionine of the PPTase is residue 5 of SEQ ID NO: 11.

### 6. DETAILED DESCRIPTION

### 6.1 Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, organic chemistry, analytical chemistry and nucleic acid chemistry described below are those well known and commonly employed in the art. As used herein, the following terms are intended to have the following meanings:

The following abbreviations are used herein:

“CoA” for coenzyme A; “TE” for thioesterase; “CAR” for carboxylic acid reductase; “ADH” for alcohol dehydrogenase; “ACP” for acyl carrier protein; “EC” means Enzyme Classification Number; CX:0 fatty acid, wherein X=8 −24 means a saturated fatty acid having X carbons (e.g., for illustrative purposes, C16:0 means hexadecanoic acid and C18:0 means octadecanoic acid); CX:1 means a monounsaturated fatty acid, wherein X=8 −24; CX:0-OH, means a saturated fatty alcohol, wherein X=8 −24 (e.g., for illustrative purposes, C14:0-OH means 1-tetradecanol and C16:0-OH means 1-hexadecanol; and C18:1-0H means 1-octadecenol); and “PPTase” is phosphopantetheinyl transferase.

“Fatty acids” are aliphatic mono carboxylic acids which may be saturated or unsaturated. As used herein a fatty acid comprises at least 8 carbon atoms. For example a saturated fatty acid has the formula CH₃(CH₂)ₓCOOH, wherein X is ≧6. Unsaturated fatty acids are of the same formula and contain one or more double bonds in the aliphatic chain.

“Fatty alcohol” as used herein refers to a long chain saturated or unsaturated hydrocarbon chain wherein the OH group attaches to the terminal carbon. As used herein a fatty alcohol comprises at least 8 carbon atoms. For example, a saturated fatty alcohol has the formula CH₃(CH₂)ₓCH₂OH, wherein x is ≧6. Unsaturated fatty alcohols are of the same formula and contain one or more double bonds in the hydrocarbon chain.

“Fatty aldehyde” as used herein refers to a saturated or unsaturated aliphatic aldehyde comprising at least 8 carbon atoms. For example, a saturated fatty aldehyde has the formula CH₃(CH₂)ₓCHO, wherein x is ≧6. Unsaturated fatty aldehydes are of the same formula and contain one or more double bonds in the aliphatic chain.

“Acyl-ACP thioesterase” (EC 3.1.2.14) used herein refers to a polypeptide having an enzymatic capability of carrying out the reaction depicted for TE in Scheme 1. Acyl-ACP thioesterases as used herein include naturally occurring (wild type) acyl-ACP thioesterases as well as non-naturally occurring engineered polypeptides generated by human manipulation.

“Alcohol dehydrogenase (ADH)” (EC 1.1.1.1) is used herein to refer to a polypeptide having an enzymatic capability of carrying out the reaction depicted for ADH in Scheme 1. Alcohol dehydrogenases as used herein include naturally occurring (wild type) alcohol dehydrogenases as well as non-naturally occurring engineered polypeptides generated by human manipulation.

“Carboxylic acid reductase (CAR)” (EC 1.2.1.30 or EC 1.2.1.3) sometimes referred to in the literature as aryl-aldehyde oxidoreductase as used herein refers to a polypeptide having an enzymatic capability of carrying out the reaction depicted for CAR in Scheme 1. Carboxylic acid reductases as used herein include naturally occurring (wild type) carboxylic acid reductases as well as non-naturally occurring engineered polypeptides generated by human manipulation. Preferred CARs of the present invention are those that require NADP/NADPH as a co-substrate.

The term “variant CAR” refers to a CAR of the present invention that is derived by manipulation from a reference CAR. Variant CARs may be constructed by modifying a DNA sequence that encodes for a wild-type CAR (e.g. a wild-type CAR depicted by SEQ ID NO: 2, SEQ ID NO: 4 or SEQ ID NO: 6).

The term “pantetheine”, IUPAC 2,4-dihydroxy-3,3-dimethyl-N-[3-oxo-3-(2-sulfanylyethylamino, propyl]butanamide and having the molecular formula of C₁₁H₂₂N₂O₄S refers to an intermediate in the pathway of coenzyme A.

A “phosphopantetheinyl transferase” (PPTase) refers to an enzyme that activates an acyl carrier protein (ACP). The phospho-pantetheine coenzyme is linked to the ACP by a phospho ester linkage. The PPTase converts the inactive apoprotein to an active holoprotein.

The terms “culturing” and “cultivation” refer to growing a population of microbial cells under suitable conditions in a liquid or solid medium. In some embodiments, culturing refers to fermentative bioconversion of a substrate to an end-product.

“Coding sequence” or “coding region” refers to that portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell (i.e. “heterologous” genes) or express native genes that are otherwise expressed at a different level.

“Recombinant host cell” or “recombinant microorganism” refers to a cell or microorganism into which has been introduced a heterologous polynucleotide or vector.

“Host cell” refers to a suitable host for an expression vector comprising DNA encoding a CAR encompassed by the invention and the progeny thereof. Host cells useful in the present invention are generally prokaryotic or eukaryotic hosts, including any transformable microorganism in which expression can be achieved.

The term “transformed” or “transformation” used in reference to a cell means a cell has a non-native nucleic acid sequence integrated into its genome or as an episomal plasmid that is maintained through multiple generations.

“Fermentable sugar” means simple sugars (monosaccharides, disaccharides and short oligosaccharides) such as but not limited to glucose, xylose, galactose, arabinose, mannose and sucrose. The term “fermentable sugar” is sometimes used interchangeably with the term “assimilable carbon source”.

“Percentage of sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Corresponding to”, “reference to”, or “relative to”, when used in the context of the numbering of a given amino acid or polynucleotide sequence refers to the numbering of the residues of a specified reference sequence when the given amino acid or polynucleotide sequence is compared to the reference sequence.

“Conversion” refers to the enzymatic conversion of the substrate to the corresponding product. “Percent conversion” refers to the percent of the substrate that is reduced to the product within a period of time under specified conditions. Thus, the “enzymatic activity” or “activity” of a polypeptide can be expressed as “percent conversion” of the substrate to the product.

“Hydrophilic Amino Acid or Residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L Ser (S), L His (H), L Glu (E), L Asn (N), L Gln (Q), L Asp (D), L Lys (K) and L Arg (R).

“Acidic Amino Acid or Residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L Glu (E) and L Asp (D).

“Basic Amino Acid or Residue” refers to a hydrophilic amino acid or residue having a side chain exhibiting a pK value of greater than about 6 when the amino acid is included in a peptide or polypeptide. Basic amino acids typically have positively charged side chains at physiological pH due to association with hydronium ion. Genetically encoded basic amino acids include L Arg (R) and L Lys (K).

“Polar Amino Acid or Residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L Asn (N), L Gln (Q), L Ser (S) and L Thr (T).

“Hydrophobic Amino Acid or Residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L Pro (P), L Ile (I), L Phe (F), L Val (V), L Leu (L), L Trp (W), L Met (M), L Ala (A) and L Tyr (Y).

“Aromatic Amino Acid or Residue” refers to a hydrophilic or hydrophobic amino acid or residue having a side chain that includes at least one aromatic or heteroaromatic ring. Genetically encoded aromatic amino acids include L Phe (F), L Tyr (Y) and L Trp (W). Although owing to the pKa of its heteroaromatic nitrogen atom L His (H) it is sometimes classified as a basic residue, or as an aromatic residue as its side chain includes a heteroaromatic ring, herein histidine is classified as a hydrophilic residue.

“Non-polar Amino Acid or Residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L Gly (G), L Leu (L), L Val (V), L Ile (I), L Met (M) and L Ala (A).

“Aliphatic Amino Acid or Residue” refers to a hydrophobic amino acid or residue having an aliphatic hydrocarbon side chain. Genetically encoded aliphatic amino acids include L Ala (A), L Val (V), L Leu (L) and L Ile (I).

“Small Amino Acid or Residue” refers to an amino acid or residue having a side chain that is composed of a total three or fewer carbon and/or heteroatom (excluding the α carbon and hydrogens). The small amino acids or residues may be further categorized as aliphatic, non-polar, polar or acidic small amino acids or residues, in accordance with the above definitions. Genetically-encoded small amino acids include L Ala (A), L Val (V), L Cys (C), L Asn (N), L Ser (S), L Thr (T) and L Asp (D).

“Hydroxyl-containing Amino Acid or Residue” refers to an amino acid containing a hydroxyl (—OH) moiety. Genetically-encoded hydroxyl-containing amino acids include L Ser (S) L Thr (T) and L-Tyr (Y).

“Conservative” amino acid substitutions or mutations refer to the interchangeability of residues having similar side chains, and thus typically involves substitution of the amino acid in the polypeptide with amino acids within the same or similar defined class of amino acids. However, as used herein, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 1 below shows exemplary conservative substitutions.

TABLE 1
Conservative Substitutions

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P, H | Other constrained (P, H) |
| N, Q, S, T | Other polar (N, Q, S, T) |
| Y, W, F | Other aromatic (Y, W, F) |
| C | None |

“Non-conservative substitution” refers to substitution or mutation of an amino acid in the polypeptide with an amino acid with significantly differing side chain properties. Non-conservative substitutions may use amino acids between, rather than within, the defined groups listed above. In one embodiment, a non-conservative mutation affects (a) the structure of the peptide backbone in the area of the substitution (e.g., proline for glycine) (b) the charge or hydrophobicity, or (c) the bulk of the side chain.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered enzyme. Deletions can be directed to the internal portions and/or terminal portions of the polypeptide. In various embodiments, the deletion can comprise a continuous segment or can be discontinuous. The term “deletion” is also used to refer to a DNA modification in which one or more nucleotides or nucleotide base-pairs have been removed, as compared to the corresponding reference, parental or “wild type” DNA.

“Insertion” refers to modification to the polypeptide by addition of one or more amino acids from the reference polypeptide. In some embodiments, the improved engineered comprise insertions of one or more amino acids to the naturally occurring polypeptide as well as insertions of one or more amino acids to other improved polypeptides. Insertions can be in the internal portions of the polypeptide, or to the carboxy or amino terminus Insertions as used herein include fusion proteins as is known in the art. The insertion can be a contiguous segment of amino acids or separated by one or more of the amino acids in the naturally occurring polypeptide. The term “insertion” is also used to refer to a DNA modification in which or more nucleotides or nucleotide base-pairs have been inserted, as compared to the corresponding reference, parental or “wild type” DNA.

“Different from” or “differs from” with respect to a designated reference sequence refers to difference of a given amino acid or polynucleotide sequence when aligned to the reference sequence. Generally, the differences can be determined when the two sequences are optimally aligned. Differences include insertions, deletions, or substitutions of amino acid residues in comparison to the reference sequence.

“Isolated polypeptide or polynucleotide” refers to a polypeptide or polynucleotide which is substantially separated from other contaminants that naturally accompany it, e.g., protein, lipids, and polynucleotides. The term embraces polypeptides and polynucleotides which have been removed or purified from their naturally-occurring environment or expression system (e.g., host cell or in vitro synthesis). Improved enzymes may be present within a cell, present in the cellular medium, or prepared in various forms, such as lysates or isolated preparations. As such, in some embodiments, the improved enzyme can be an isolated polypeptide.

“Heterologous” polynucleotide, gene, promoter, or polypeptide refers to any polynucleotide, gene, promoter, or polypeptide that is introduced into a host cell by laboratory techniques, and includes a polynucleotide, gene, promoter, or polypeptide that is removed from a host cell, subjected to laboratory manipulation, and then reintroduced into a host cell.

“Endogenous” polynucleotide, gene, promoter or polypeptide refers to any polynucleotide, gene, promoter or polypeptide that is in the cell and was not introduced into the cell using laboratory or recombinant techniques.

“Improved enzyme property” refers to a polypeptide that exhibits an improvement in any enzyme property as compared to a reference polypeptide. For the engineered polypeptides described herein, the comparison is generally made to the wild-type enzyme. Enzyme properties for which improvement is desirable include, but are not limited to, enzymatic activity, thermal stability, pH activity profile, refractoriness to inhibitors, e.g., feedback inhibition, product inhibition, and substrate inhibition, as well as increased stability and/or activity in the presence of additional components present in, added to, or formed within the aqueous nutrient medium or within the recombinant host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding enzymes may be codon optimized for optimal production from the host organism selected for expression.

“Preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG Codon Preference, Genetics Computer Group Wisconsin Package; CodonW, John Peden, University of Nottingham; McInerney, J. O,1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTs), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Hybridization stringency” relates to such washing conditions of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA; with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tm as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Other high stringency hybridization conditions, as well as moderately stringent conditions, are described in the references cited above.

“Control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” and “operably associated” are defined herein as a configuration in which a control sequence is appropriately placed at a position relative to the coding sequence of the DNA sequence such that the control sequence directs the expression of a polynucleotide and/or polypeptide.

“Promoter sequence” is a nucleic acid sequence that is recognized by a host cell for expression of the coding region. The control sequence may comprise an appropriate promoter sequence. The promoter sequence contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

As used herein “a”, “an”, and “the” include plural references unless the context clearly dictates otherwise.

The term “comprising” and its cognates are used in their inclusive sense; that is, equivalent to the term “including” and its corresponding cognates.

### 6.2 Host Cells Useful in the Disclosed Process

In some embodiments, the host cell is a eukaryotic cell. Suitable eukaryotic host cells include, but are not limited to, fungal cells and algal cells. Some preferred fungal host cells are yeast cells and filamentous fungal cells.

The filamentous fungal host cell may be a cell of a species of, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, Volvariella, or teleomorphs, synonyms or taxonomic equivalents thereof.

In some embodiments of the invention, the filamentous fungal host cell is an Aspergillus species, a Chrysosporium species, a Corynascus species, a Fusarium species, a Humicola species, a Myceliophthora species, a Neurospora species, a Penicillum species, a Tolypocladium species, a Tramates species, or Trichoderma species. In some embodiments of the invention, the Trichoderma species is T. longibrachiatum, T. viride, Hypocrea jecorina or T. reesei; the Aspergillus species is A. awamori, A. funigatus, A. japonicus, A. nidulans, A. niger, A. aculeatus, A. foetidus, A. oryzae, A. sojae, and A. kawachi; the Chrysosporium species is C. lucknowense; the Fusarium species is F. graminum, F. oxysporum and F. venenatum; the Neurospora species is N. crassa; the Humicola species is H. insolens, H. grisea, and H. lanuginosa; the Myceliophthora species is M. thermophilic; the Penicillum species is P. purpurogenum, P. chrysogenum, and P. verruculosum; the Thielavia species is T. terrestris; and the Trametes species is T. villosa and T. versicolor.

In the present invention, a yeast host cell may be a cell of a species of, but not limited to Candida, Hansenula, Saccharomyces, Schizosaccharomyces, Pichia, Kluyveromyces, and Yarrowia. In some embodiments, the yeast host cell may be a cell of a species of Saccharomyces, Pichia, Candida or Yarrowia. In some embodiments of the invention, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, Candida Candida krusei, and Yarrowia lipolytica. Particularly useful Yarrowia lipolytica strains include but are not limited to DSMZ 1345, DSMZ 3286, DSMZ 8218, DSMZ 70561, DSMZ 70562, and DSMZ 21175.

In some embodiments of the invention, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative and gram-variable bacterial cells. The host cell may be a species of, but not limited to Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmun, Streptomyces, Streptococcus, Synnecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some preferred embodiments, the host cell may be a species of, but not limited to Agrobacterium, Arthrobacter, Bacillus, Clostridium, Corynebacterium, Escherichia, Erwinia, Geobacillus, Klebsiella, Lactobacillus, Mycobacterium, Pantoea, Streptomyces and Zymomonas.

In some embodiments, the bacterial host strain is an industrial strain. Numerous bacterial industrial strains are known and suitable in the present invention. In some embodiments of the invention, the bacterial host cell is of the Bacillus species, e.g., B. thuringiensis, B. anthracis, B. megaterium, B. subtilis, B. lentus, B. circulans, B. pumilus, B. lautus, B. coagulans, B. brevis, B. firmus, B. alkaophius, B. lichenifonnis, B. clausii, B. stearothermophilus, B. halodurans and B. amyloliquefaciens. In some embodiments the host cell will be an industrial Bacillus strain including but not limited to B. subtilis, B. pumilus, B. lichenifonnis, B. clausii, B. stearothermophilus and B. amyloliquefaciens.

In some embodiments, the bacterial host cell is of the Escherichia species, e.g., E. coli.

In some embodiments, the bacterial host cell is of the Erwinia species, e.g., E. uredovora, E. carotovora, E. ananas, E. herbicola, E. punctata, and E. terreus.

In some embodiments, the bacterial host cell is of the Pantoea species, e.g., P. citrea, and P. agglomerans.

In some embodiments, the bacterial host cell is of the Streptomyces species, e.g., S. ambofaciens, S. achromogenes, S. avermitilis, S. coelicolor, S. aureofaciens, S. aureus, S. fungicidicus, S. griseus, and S. lividans.

In some embodiments, the bacterial host cell is of the Zymomonas species, e.g., Z. mobilis, and Z. lipolytica.

Strains which may be used in the practice of the invention including both prokaryotic and eukaryotic strains, and these are readily accessible to the public from a number of culture collections such as American Type Culture Collection (ATCC), Deutsche Sammlung von Mikroorganismen and Zellkulturen GmbH (DSMZ) (German Collection of Microorganisms and Cell Culture), Centraalbureau Voor Schimmelcultures (CBS), and Agricultural Research Service Patent Culture Collection, Northern Regional Research Center (NRRL).

In some particular embodiments, recombinant microorganisms encompassed by the inventions are derived from strains of Escherichia coli, Bacillus, Saccharomyces, Streptomyces and Yarrowia.

The recombinant microorganisms that are capable of producing fatty alcohols as encompassed by the invention will include heterologous genes encoding a carboxylic acid reductase. In some embodiments, the recombinant microorganisms will additionally comprise one or more heterologous genes selected from the group of acyl-ACP thioesterases, alcohol dehydrogenases and/or PPTases as further described below.

The present disclosure provides a process for conversion of carbon sources assimilable by recombinant microorganisms to fatty alcohols. Microorganisms have evolved efficient processes for the conversion of carbon sources to fatty acids. The presently disclosed process exploits that efficiency by diverting the fatty acids so produced to long chain fatty alcohols by metabolic engineering of the host microorganism. In one aspect, this is accomplished by developing a pathway within a recombinant host cell, which pathway is depicted in Scheme 1 below:

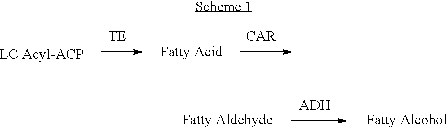

In this scheme LC Acyl-ACP refers to a long chain fatty acid (e.g., C8−C24) bound to an acyl carrier protein by a thioester bond. The enzymes of the pathway depicted in Scheme 1 include an acyl ACP thioesterase (TE), a carboxylic acid reductase (CAR), and a ketoreductase/alcohol dehydrogenase (ADH). In a preferred embodiment of the invention, the CAR will be heterologous to the host cell. In some embodiments of the invention, the recombinant microorganism will include at least one additional heterologous gene encoding a polypeptide selected from the set of enzymes comprising: acyl-ACP thioesterase (TE) and dehydrogenase/ketoreductase (ADH). In some embodiments, the pathway of scheme 1 is the preferred pathway in bacterial host cells and particularly E. coli host cells.

In another scheme of the invention, the fatty acid may be derived from a source other than a LC Acyl-ACP; for example, the hydrolysis of triglycerides and/or phospholipids.

In a particular aspect, the recombinant microorganism further comprises a gene expressing a phosphopantetheinyl transferase polypeptide capable of attaching a pantetheine moiety to the carboxylic acid reductase polypeptide (CAR) depicted in Scheme 1 above.

### 6.3 Enzymes Useful in the Disclosed Process

### Carboxylic Acid Reductase (CAR)

As disclosed herein, it has been discovered that carboxylic acid reductases are capable of reducing a fatty acid to the corresponding aldehyde, as depicted below in Scheme 2:

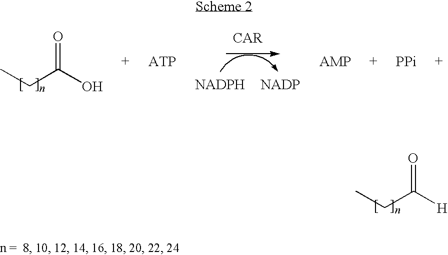

Carboxylic acid reductases (CAR) are unique ATP- and NADPH-dependent enzymes that reduce carboxylic acids, such as fatty acids to the corresponding aldehyde. CARs are multi-component enzymes comprising a reductase domain; an adenylation domain and a phosphopantetheine attachment site. As disclosed herein, fatty acids, such as those fatty acids comprising 8 to 24 carbon atoms and particularly those fatty acids comprising 12 carbon atoms (dodecanoic acid) to 18 carbon atoms (stearic acid)) may be reduced by a carboxylic acid reductase of the invention such as those having at least 85%, at least 90%, at least 93%, at least 95%, at least 96%, at least 97%, at least 98%, at least 99%, or even 100% sequence identity to the CAR of Mycobacterium sp. JLS, as illustrated in SEQ ID NO: 4; a carboxylic acid reductase having at least 85%, at least 90%, at least 93%, at least 95%, at least 96%, at least 97%, at least 98%, at least 99%, or even 100% sequence identity to the CAR of Nocardia sp. NRRL5646 as illustrated in SEQ ID NO: 2 or SEQ ID NO: 9; a carboxylic acid reductase having at least 85%, at least 90%, at least 93%, at least 95%, at least 96%, at least 97%, at least 98%, at least 99%, or even 100% sequence identity to the CAR of Streptomyces griseus as illustrated in SEQ ID NO: 6 or SEQ ID NO: 10.

In some embodiments, the carboxylic acid reductase has at least 85%, at least 90%, at least 93%, at least 95%, at least 96%, at least 97%, at least 98%, at least 99% or even 100% sequence identity to a CAR protein comprising the 4-mer GDIH appended at the amino terminus (SEQ ID NOs: 9 and 10). Reference is also made to the Nocardia sp. CAR disclosed in U.S. Pat. No. 6,261,814.

The present invention also encompasses variant CARs. The variant may comprise at least 90% (e.g., at least 93%, at least 95%, at least 96%, at least 97%, at least 98%, or at least 99%) sequence identity to SEQ ID NO: 2, SEQ ID NO: 4 or SEQ ID NO: 6. In some embodiments, the variant CAR comprises at least 90% (e.g., at least 93%, at least 95%, at least 96%, at least 97%, at least 98% and at least 99%) sequence identity with SEQ ID NO: 4 and a substitution of an amino acid at a position corresponding to position 8270, A271, K274, A275, P467, Q584, E626, and/or D701 when aligned with SEQ ID NO: 4. In some embodiments, the variant CAR may include an amino acid sequence that is at least 85%, (e.g., at least 90%, at least 93%, at least 95%, at least 96%, at least 97%, at least 98% and at least 99%) identical to SEQ ID NO: 4 and an amino acid substitution corresponding to R270W, A271W, K274(G/N/V/I/W/L/M/Q/S), A275F, P467S, Q584R, E626G, D701G, K274L/A369T/L380Y, K274LN358H/E845A, K274M/T282K, K274Q/T282Y, K274S/A715T, K274W/L380G/A477T, K274W/T282E/L380V, K274W/T282Q, K274W/V358R and/or R43c/K274I in SEQ ID NO: 4. In some embodiments, the variant CAR will comprise an amino acid substitution at position K274 and one or more (e.g., 1, 2 or 3) further amino acid substitutions when the variant is aligned with SEQ ID NO: 4. In some embodiments, the CAR activity of the variant will be greater than the CAR activity of the reference or parent sequence. CAR activity can be determined for example by the assays described in examples below.

In some embodiments, a variant may encompass additional amino acid substitutions at positions other than those listed above including, for example, variants with one or more conservative substitutions. Examples of conservative substitutions are disclosed herein above. In some embodiments conservatively substituted variations of a CAR will include substitutions of a small percentage, typically less than 5%, more typically less than 2%, and often less than 1% of the amino acids of the polypeptide sequence.

As noted below, intracellular expression of a carboxylic acid reductase of the invention, will lead to production not only of the fatty aldehyde but also the corresponding fatty alcohol. This is the result of alcohol dehydrogenase activity within the recombinant host cell. Reference is made to Scheme 3 below. In some embodiments, the process will result in the production of fatty alcohols comprising C8, C10, C12, C14, C16, C18, C20, C22 and C24 carbons in length.

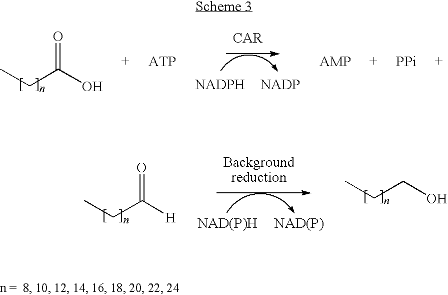

In certain embodiments of the present disclosure, the recombinant host cell expresses a carboxylic acid reductase that, as compared to its parent or the wild-type enzyme has a lower Kₘ for each of its carboxylic acid and ATP substrates, has an increased Vₘₐₓ and/or k_{cat} or a different carbon chain length profile with respect to the fatty aldehyde products it catalyzes for the reaction depicted in Schemes 2 and 3 or is more resistant to inhibition by increased concentrations of its carboxylic acid and ATP substrates or by increased concentrations of the fatty aldehyde, AMP, and pyrophosphate products of the reaction depicted in Schemes 2 and/or 3.

### Phosphopantetheinyl Transferase (PPTase)

In some embodiments, the recombinant microorganism of the invention will express a phosphopantetheinyl transferase (PPTase) polypeptide which is capable of attaching a pantetheine moiety to the CAR. In some embodiments, the PPTase will be a transferase from a bacterial organism. In some embodiments, the transferase will be a Nocardia PPTase, (such as, but not limited to a PPTase derived from N. iowensis or N. farcinica); a Mycobacterium PPTase (such as, but not limited to a PPTase derived from M. abscessus (ATCC 19977), M. sp., MCS, M. vanbaalenii, M avium, M. bovis, M. marinum or M. smegmatis); a Rhodococcus PPTase (such as, but not limited to PPTases derived from R. jostii, R. opacus, or R. erythropolis) a Streptomyces PPTase (such as, but not limited to S. verticillus) or a Gordonia PPTase (such as, but not limited to a PPTase derived from G. bronchialis). PPTases derived from these organisms are known in the art and reference is made to Venkitasubramanian, P. et al 2007, J. Biological Chemistry Vol. 282 pp 478-485 and Sanchez, C. et al., 2001 Chem. Biol. Vol. 8 pp 725-738. In some embodiments, the PPTase will have at least 50%, at least 60%, at least 70%, at least 80%, at least 85%, at least 90%, at least 91%, at least 92%, at least 93%, at least 95%, at least 96%, at least 97%, at least 98%, at least 99% and even 100% sequence identity to SEQ ID NO: 8 or SEQ ID NO: 11.

Some host microorganisms have more than one PPTase. For example E. coli is believed to have three classes of PPTases and these PPTases have been classified depending on their sequence identity and substrate spectrum. In addition, two PPTases have been identified in Bacillus subtilis. Preferred PPTases encompassed by this invention are those that are involved in the modification of fatty acid ACP. In some embodiments the PPTase will be heterologous to the host microorganism, and in other embodiments, the PPTase may be derived from the host microorganism but will be over expressed by genetic manipulation in the host.

### Acyl ACP Thioesterase

Acyl ACP thioesterases catalyze the hydrolysis of acyl-ACP (i.e. acylated Acyl Carrier Protein) that are intermediates in the biosynthesis of fatty acids, as depicted in Scheme 4 below, wherein n preferably is 10-18:

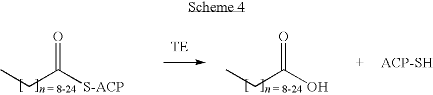

The methods of the present invention preferably utilize an endogenous TE in the process of producing a fatty alcohol. However, host cells may be manipulated to include a heterologous TE. For example, host cells may over-express an acyl ACP thioesterase that has been manipulated and then introduced into the host cell. In some embodiments, the acyl-ACP thioesterase is obtained from Escherichia coli, Cuphea hookeriana, Umbellularia california, Cinnamonum camphorum, Arabidipsis thaliana, Brassica junicea and Bradyrhiizobium japonicum acyl-ACP thioesterases. Genes (such as tesA, tesB, fatB, fatA, fatA1 and the like) coding for TEs are known in the art and available from public database such as NCBI and GenBank. Examples include but are not limited to Accession numbers AAC73596; Q41635; AAC72881; AAC72883; AAC73555; POADA1; and Q39473.

In certain embodiments of the present invention, the recombinant host cell expresses a heterologous acyl-ACP thioesterase that, as compared to its parent or the wild-type enzyme has a lower Kₘ for its thioester substrate, has an increased Vₘₐₓ and/or k_{cat}, or a different carbon chain length profile with respect to the fatty acid products it catalyzes for the reaction depicted in Scheme 4 above, or is more resistant to inhibition by increased concentrations of its thioester substrate or by increased concentrations of the carboxylic acid and ACP-SH products of the reaction depicted in Scheme 4.

### Alcohol Dehydrogenase/Ketoreductase (ADH)

Alcohol dehydrogenases (ketoreductases) catalyze the conversion of an aldehyde to the corresponding alcohol for example, as depicted in Scheme 5:

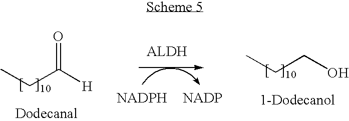

In this reaction the aldehyde, dodecanal and the corresponding alcohol, 1-dodecanol are representative of a species of a genus of various aldehyde substrates. Other preferred aldehyde reactions include the conversion of a C8, C12, C14, C16, C18, C20, C22 and C24 aldehyde to the corresponding saturated or unsaturated fatty alcohol. This reaction is energetically favorable and occurs without activation of the substrate. The method of the present invention preferably utilizes an endogenous ADH in the process of producing a fatty alcohol. However, host cells may be manipulated to include a heterologous ADH. For example, host cells may over-express an ADH that has been manipulated and then introduced into the host cell. In some embodiments, the alcohol dehydrogenase is an E. coli ADH (genes coding for E. coli ADHs include but are not limited to dkgA and B; adhP, and yhdH). In some embodiments the ADH is a Yarrowia lipolytica ADH such as but not limited to ADH1-4 and also NCIB accession numbers Q9UWO8 (AAD51737.1); Q9UWO6 (AAD51739.1); Q9UWO7 (AAD51738.1) and CAG79261.

In certain embodiments of the present disclosure, the recombinant host cell expresses a heterologous alcohol dehydrogenase that, as compared to its parent or the wild-type enzyme has a lower Kₘ for each of its long chain aldehyde substrate, has an increased Vₘₐₓ and/or k_{cat} or a different carbon chain length profile with respect to the fatty alcohol products it catalyzes for the reaction depicted in Scheme 5 above, or is more resistant to inhibition by increased concentrations of its fatty aldehyde substrate or by increased concentrations of the fatty alcohol product.

The recombinant host cells of the present invention may also comprise mutations that lead to an increase in the levels of fatty acid produced by the host cell as well as mutations resulting in a decreased rate of utilization of fatty acids in competing pathways, e.g., lipid synthesis, fatty acid β-oxidation, sphingolipid biosynthesis, and protein acylation. Additional mutations that may be introduced into the recombinant host cells of the invention include those enhancing processes that result in the extracellular accumulation of the fatty alcohols synthesized in the recombinant host cells of the disclosure. In certain embodiments, the recombinant host cells comprise combinations of mutations that, collectively, e.g., provide increased levels of fatty acid production coupled with decreased rates of utilization of those fatty acids in competing pathways, as well as increased extracellular accumulation of long chain fatty alcohols.

In certain embodiments, the recombinant host cells of the invention comprise mutations eliminating or selectively repressing metabolic regulatory pathways, e.g., feedback inhibition by long chain fatty acids, whereby the biosynthesis of fatty acids is repressed while the production of enzymes for fatty acid catabolism are induced, or glucose repression of pathways for the transport and use of alternative carbon sources, such as galactose.

### 6.4 Nucleic Acids, Genes and Vectors Useful in the Disclosed Process

In another embodiment, the present disclosure provides DNA constructs, vectors and polynucleotides encoding the enzymes (e.g., CARs) of the invention. Polynucleotides may be operably linked to one or more heterologous regulatory sequences that control gene expression to create a recombinant polynucleotide capable of expressing the polypeptide. Expression constructs containing a heterologous polynucleotide encoding the polypeptides of the invention can be introduced into appropriate host cells to express the corresponding polypeptide. Because of the knowledge of the codons corresponding to the various amino acids, availability of a protein sequence provides a description of all the polynucleotides capable of encoding the subject polypeptide. The degeneracy of the genetic code, where the same amino acids are encoded by alternative or synonymous codons allows an extremely large number of nucleic acids to be made, all of which encode the enzymes encompassed by the invention. Thus, having identified a particular amino acid sequence, those skilled in the art could make any number of different nucleic acids. In some embodiments, the codons are selected to fit the host cell in which the protein is being produced. For example, preferred codons used in bacteria are used to express the gene in bacteria and preferred codons used in yeast are used for expression in yeast. In some embodiments, the polynucleotide comprises a nucleotide sequence encoding a CAR polypeptide with an amino acid sequence that has at least about 80%, 85%, 86%, 87%, 88%, 89%, 90%, 91%, 92%, 93%, 94%, 95%, 96%, 97%, 98%, or 99% or more sequence identity to SEQ ID NOs: 2, 4, 6, 9 or 10. In some embodiments, the polynucleotide will be SEQ ID NO: 1, SEQ ID NO: 3 or SEQ ID NO: 5. In some embodiments, the polynucleotide sequence will have at least 90%, at least 93%, at least 95%, at least 96%, at least 97%, at least 98% and at least 99% sequence identity to SEQ ID NOs: 1, 3 or 5. In some embodiments, the polynucleotide will be a sequence that hybridizes to SEQ ID NO: 1, SEQ ID NO: 3, or SEQ ID NO: 5 under high stringency conditions.

In some embodiments the polynucleotide encoding a PPTase useful in a process of producing fatty alcohols according to the invention will have at least 50%, at least 60%, at least 70%, at least 80%, at least 85%, at least 90%, at least 93%, at least 95%, at least 96%, at least 97%, at least 98%, at least 99% and even 100% sequence identity to SEQ ID NO: 7.

An isolated polynucleotide encoding a polypeptide encompassed by the invention may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updates to 2006.

One skilled in the art is well aware of techniques which may be used to generate polynucleotides which code for variant CARs and these techniques include but are not limited to classical and/or synthetic DNA shuffling techniques. Classical DNA shuffling generates variant DNA molecules by in vitro homologous recombination from random fragmentation of a parent DNA followed by reassembly using ligation and/or PCR, which results in randomly introduced point mutations. A resulting library can in turn be screened and further shuffled. Synthetic DNA shuffling may also be used wherein a plurality of oligonucleotides are synthesized which collectively encode a plurality of mutations to be combined. Recombination-based evolution may further be complemented by protein sequence activity relationships (e.g., ProSAR), which incorporates statistical analysis in targeting amino acid residues for mutational analysis. See e.g., Fox et al., Nature Biotechnology 25: 338-344 92007).

The polynucleotides encoding polypeptides encompassed by the invention are operably linked to a promoter and optionally other regulatory sequences. Suitable promoters include constitutive promoters, regulated promoters and inducible promoters.

For bacterial host cells, suitable promoters for directing transcription of the nucleic acid constructs of the present disclosure include the promoters obtained from E. coli. Other suitable promoter may be the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus lichenifonnis penicillinase gene (penP), Bacillus subtilis xy1A and xy1B genes, and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Scientific American, 1980, 242:74-94; and in Sambrook et al., supra.

For filamentous fungal host cells, suitable promoters for directing the transcription of the nucleic acid constructs of the present disclosure include but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787).

In a yeast host, useful promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase; (TEF1) promoter. Other useful promoters for yeast host cells are described by Romanos et al., 1992, Yeast 8:423-488.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention. Exemplary terminators for yeast host cells can be obtained from the genes for Saccharomyces cerevisiae enolase, Saccharomyces cerevisiae cytochrome C(CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are described by Guo and Sherman, 1995, Mol Cell Bio 15:5983-5990. The control sequence may also be a signal peptide coding region that codes for an amino acid sequence linked to the amino terminus of a polypeptide and directs the encoded polypeptide into the cell's secretory pathway.

The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Exemplary signal peptides for yeast host cells can be from the genes for Saccharomyces cerevisiae alpha-factor and Saccharomyces cerevisiae invertase. Other useful signal peptide coding regions are described by Romanos et al., 1992, supra. The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

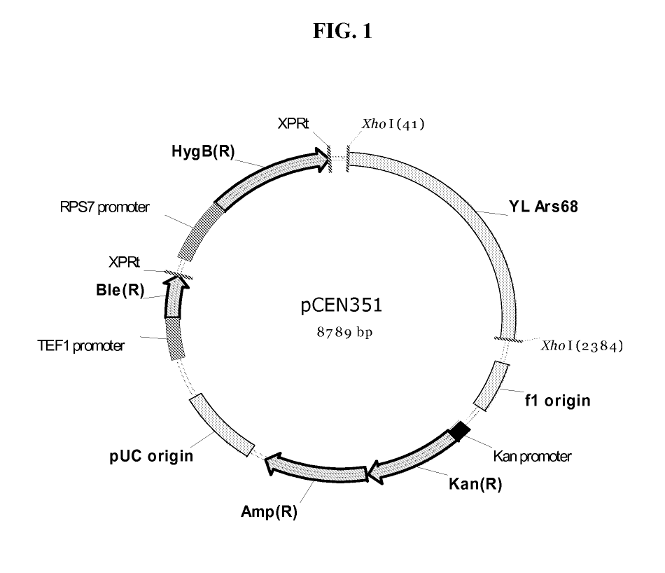

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids. The expression vector will typically include a selectable marker such as but not limited to antibiotic resistance such as ampicillin, kanamycin, chloramphenicol or tetracycline resistance. Suitable markers for yeast host cells are ADE2, HIS3, LEU2, LYS2, MET3, TRP1, and URA3. Examples of expression vectors which may be useful in the present invention are commercially available for example from Sigma-Aldrich Chemicals, St. Louis Mo. and Stratagene, LaJolla Calif., and plasmids which are derived from pBR322 (Gibco BRL), pUC (Gibco BRL), pREP4, pCEP4 (Invitrogen) or pPoly (Lathe et al., 1987, Gene 57:193-201). In one embodiment, the present disclosure provides an autonomous replicating plasmid for expression of heterologous genes in Yarrowia and particularly in Y. lipolytica. This plasmid vector (pCEN351; FIG. 1) was engineered with two antibiotic selection marker cassettes for resistance to hygromycin and phleomycin (Hyg B^{R} or Ble^{R}, respectively). In this embodiment, expression of each cassette is independently regulated by a strong, constitutive promoter isolated from Y. lipolytica: pTEF 1 for Ble^{R} expression and pRPS7 for HygB^{R} expression. When this plasmid, pCEN351, was transformed into Y. lipolytica, it conferred resistance to both hygromycin and phleomycin, validating the functionality of each cassette. This plasmid and the two selection markers enable expression of heterologous genes useful for fatty alcohol production in yeast, inter alia, Y. lipolytica. The antibiotic resistance cassettes constructed above are also useful for gene disruptions in Y. lipolytica. In such embodiments, for example, the antibiotic resistance cassettes are used to perform knockouts of genes involved in degradation of free fatty acids and fatty acyl-CoA compounds. Such gene disruption can be performed by homologous recombination, an established method in Y. lipolytica (see e.g. EP 0 138 508 B1, U.S. Pat. No. 4,889,741 and U.S. Pat. No. 5,071,764, each of which is hereby incorporated by reference in its entirety).

In some embodiments a vector according to the invention will comprise a polynucleotide sequence coding for a CAR as described herein. In other embodiments, a vector may include a polynucleotide coding for a PPTase as described herein, for example a PPTase having at least 80% sequence identity to the PPTase of SEQ ID NO: 8. In some embodiments the polynucleotide coding for the PPTase and the polynucleotide coding for the CAR are both on the same plasmid. In some preferred embodiments, the vector is a plasmid such as pCEN351 which can be adapted for over-expression of the fatty alcohol pathway genes identified herein, by replacing one of the selection markers with a gene(s) of interest. For example, the Ble^{R} gene can be replaced with different genes encoding enzymes for reduction of fatty acids (e.g. TE).

Methods for the transformation of Y. lipolytica strain PO1 g (Yeastern Biotech) are known in the art. In other embodiments, modified procedures for the transformation Y. lipolytica strains have been developed. In certain embodiments, the expression vectors of the present disclosure are integrated into the chromosome of the recombinant host strain and comprise one or more heterologous genes operably associated with a promoter useful for production of long chain fatty alcohols. In other embodiments, the expression vectors are extrachromosomal replicative DNA molecules, e.g. plasmids, that are found in low copy number (e.g. 1-10 copies per genome equivalent) or in high copy number (more than 10 copies per genome equivalent).

In certain aspects, the present disclosure is directed to expression vectors comprising heterologous genes useful for the production of fatty alcohols (e.g., C8, C10, C12, C14, C16, C18, C20, C22 and C24), wherein each heterologous gene is operably linked with a promoter that may be independently selected to provide a desired level of expression of the heterologous gene.

### 6.5 Culture Conditions and Long Chain Fatty Alcohol Recovery

In certain embodiments of the present disclosure, the recombinant host strain comprising at least one heterologous gene encoding a CAR is cultured in an aqueous nutrient medium comprising an assimilable source of carbon, whereby long chain fatty alcohols are produced. The individual components of such media are available from commercial sources, e.g., under the Difco™ and BBL™ trademarks.

In one non-limiting example, the aqueous nutrient medium is a “rich medium” comprising complex sources of nitrogen, salts, and carbon, such as YP medium, comprising 10 g/L of peptone and 10 g/L yeast extract of such a medium.

In other non-limiting embodiments, the aqueous nutrient medium comprises a mixture of Yeast Nitrogen Base (Difco) in combination supplemented with an appropriate mixture of amino acids, e.g. SC medium. In particular aspects of this embodiment, the amino acid mixture lacks one or more amino acids, thereby imposing selective pressure for maintenance of an expression vector within the recombinant host strain.

Fermentation of the recombinant host strain comprising at least one heterologous gene useful for production of long chain fatty alcohols is carried out under suitable conditions and for a time sufficient for production of long chain fatty alcohols. Many references are available for the culture and production of many cells, including cells of bacterial, yeast and fungal cells. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue (1998) from Sigma-Aldrich, Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference.

In some embodiments, cells expressing the CAR and/or other recombinant enzymes of the invention are grown under batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady sate growth conditions. Methods for modulating nutrients and growth factors for continuous fermentation processes as well as techniques for maximizing the rate of product formation are well known in the art of industrial microbiology.

In various aspects, such culturing or fermentations are carried out at a temperature within the range of from about 10° C. to about 80° C., from about 15° C. to about 75° C., from about 15° C. to about 65° C., from about 20° C. to about 60° C., from about 20° C. to about 55° C., from about 20° C. to about 50° C., and from about 25° C. to about 40° C. In other embodiments, the fermentation is carried out for a period of time within the range of from about 8 hours to about 240 hours, from about 10 hours to about 192 hours, from about 20 hours to about 96 hours, or from about 24 to about 72 hours. In other embodiments, culturing is carried out at a pH range of 3.5 to 7.5 (such as pH 4.0 to 7.0; pH 4.5 to 7.0 and pH 5.0 to 7.0).

Carbon sources useful in the aqueous fermentation medium or broth of the disclosed process in which the recombinant microorganisms are grown are those assimilable by the recombinant host strain. Assimilable carbon sources are available in many forms and include renewable carbon sources and the cellulosic and starch feedstock substrates obtained there from. Such examples include for example monosaccharides, disaccharides, oligosaccharides, saturated and unsaturated fatty acids, succinate, acetate and mixtures thereof. Further carbon sources include, without limitation, glucose, galactose, sucrose, xylose, fructose, glycerol, arabinose, raffinose, lactose, maltose, and mixtures thereof. In one aspect of this embodiment, the fermentation is carried out with a mixture of glucose and galactose as the assimilable carbon source. In another aspect, fermentation is carried out with glucose alone to accumulate biomass, after which the glucose is substantially removed and replaced with an inducer, e.g., galactose for induction of expression of one or more heterologous genes involved in fatty alcohol production. In still another aspect, fermentation is carried out with an assimilable carbon source that does not mediate glucose repression, e.g., raffinose, to accumulate biomass, after which the inducer, e.g., galactose, is added to induce expression of one or more heterologous genes involved in fatty alcohol production. In some preferred embodiments, the assimilable carbon source is from cellulosic and starch feedstock derived from but not limited to, wood, wood pulp, paper pulp, grain, corn stover, corn fiber, rice, paper and pulp processing waste, woody or herbaceous plants, fruit or vegetable pulp, distillers grain, grasses, rice hulls, wheat straw, cotton, hemp, flax, sisal, corn cobs, sugar cane bagasse, switch grass and mixtures thereof.

In certain aspects, the invention relates to a process for the biologically-derived production of fatty alcohols which comprises culturing the recombinant microbial host cell said host cell including a polynucleotide encoding a heterologous CAR polypeptide and culturing the recombinant microorganism in an aqueous nutrient medium comprising an assimilable source of carbon under suitable culture conditions for a sufficient period of time to allow the production the fatty alcohols and further recovering the fatty alcohols. In certain embodiments the recombinant host cell is a yeast such as but not limited to a Saccharomyces cerevisiae or Yarrowia lipolytica. In other embodiments, the recombinant host cell is a bacterial cell, such as but not limited to cells of E. coli or Bacillus sp.

In some embodiments the invention relates to a process for the biologically-derived production of fatty alcohols in a yeast cell which comprises a) culturing a recombinant yeast cell comprising a polynucleotide which encodes a CAR as herein described in an aqueous nutrient medium comprising an assimilable carbon source derived from a cellulosic or starch feedstock under suitable culture conditions for a sufficient period of time to allow expression of the CAR; b) producing the fatty alcohol and c) recovering the fatty alcohol. In some embodiments, the yeast cell is a Saccharomyces strain (e.g., S. cerevisiae) or a Yarrowia strain (e.g., Y. lipolytica).

While the fatty alcohols produced by the process of the invention include both saturated and unsaturated fatty alcohols, including monounsaturated fatty alcohols, with one or more double bonds (e.g., Δ9-hexadecenol), some preferred fatty alcohols include octan-1-ol, decan-1-ol, dodecan-1-ol, tetradecan-1-ol, hexadecane-1-ol, octadecan-1-ol, and icosan-1-ol. In a most preferred embodiment, the produced fatty alcohols will include C14, C16 and C18 fatty alcohols such as tetradecan-1-ol, hexadecane-1-ol, and octadecan-1-ol.

In some embodiments of the process encompassed by the invention, the production of fatty alcohols (C8-C24) from a recombinant host cell will be in the range of about 2 mg/L to 250 g/L; about 2 mg/L to 200 g/L; about 5 mg/L to 150 g/L; about 10 mg/L to 150 g/L; and about 50 mg/L to 100 g/L of fermentation media. In some embodiments, the amount of fatty alcohol produced is greater than 500 mg/L, greater than 1.0 g/L, greater than 5.0 g/L, greater than 10.0 g/L greater than 25 g/L greater than 50 g/L, greater than 75 g/L, greater than 100 g/L, greater than 150 g/L and also greater than 175 g/L of media. For example, in some embodiments, the amount of fatty alcohol produced by a recombinant yeast cell according to the invention will be at least 2 mg/L, also at least 5 mg/L, also at least 10 mg/L and also at least 1 g/L of media.

In some embodiments, the production of fatty alcohols by the process of the invention will be in the range of about 0.1 mg/g to 10 g/g dry cell weight (DCW); in the range of about 100 mg/g to 10 g/g DCW, in the range of 500 mg/g to 10 g/g DCW and also in the range of 1 g/g to 5 g/g DCW.

In some embodiments the production of fatty alcohols having C8 to C20 carbons in length will comprise at least 85%, at least 90%, at least 93%, at least 95%, at least 97% and at least 98% of the total isolated fatty alcohols. In some embodiments, the production of fatty alcohols having C10 to C18 carbons in length will comprise at least 85%, at least 88%, at least 90%, at least 93%, and at least 95% of the total produced isolated fatty alcohols.

Recovering when used in reference to “recovering” or “isolating” the fatty alcohols produced by a recombinant microorganism according to the invention includes, but is not limited to, recovering the fatty alcohols from the recombinant cells or recovering the fatty alcohols from the extracellular environment such as the culture media. In some embodiments, the fatty alcohols may be produced and released (e.g., secreted) from the recombinant cells into the culture or fermentation media. In other embodiments the recombinant or host cell is lysed prior to separation of the produced fatty alcohols. In some embodiments, the recovered fatty alcohols are further purified. Purification does not require absolute purity but is a relative term and means that the recovered fatty alcohols may be further separated from other cellular components such as but not limited to other proteins, hydrocarbons and lipids.

In certain aspects of the disclosure, long chain fatty alcohols are isolated by solvent extraction of the fermentation medium with a suitable water-immiscible solvent. Phase separation followed by solvent removal provides the long chain fatty alcohol which may then be further purified and fractionated using methods and equipment known in the art. In other aspects of the disclosure, the long chain fatty alcohols coalesce to form a water-immiscible phase that can be directly separated from the nutrient aqueous medium either during the fermentation or after its completion, or precipitate from the aqueous medium and can be separated by filtration or solvent extraction. Reference is made to Can J. Biochem Physiol., 1959, 37:911-7, J. Biol. Chem., 1957, 226, 497-509 and examples herein below.

In some embodiments the fatty alcohols will be further reduced to the corresponding alkanes. Means for reducing fatty alcohols are well known in the art. In one example, the fatty alcohol may be dehydrated to a corresponding alkene and then the alkene is hydrogenated to the corresponding alkane.

In another embodiment, the invention relates to a method of catalytically reducing a fatty acid substrate to a corresponding C8-C24 carbon containing fatty aldehyde comprising mixing an effective amount of a CAR polypeptide according to the invention with a fatty acid substrate and cofactors selected from the group of ATP and NADPH and incubating the mixture for a period of time and under conditions suitable to achieve reduction of the substrate to the corresponding fatty aldehyde. In some embodiments the fatty aldehyde is reduced to the corresponding fatty alcohol.

In some embodiments the fatty alcohol may be further converted to a fatty ester by either chemical or enzymatic means (such as by the use of lipases). Methods of conversion to fatty esters are well known in the art.

### 6.6 Post Production and Compositions

The fatty alcohols produced by the process described herein can either be used directly or further processed for example but not limited to use in the production of fuels, chemicals, lubricants, cosmetics and fuel blends. Fuels include gasoline, diesel, and jet fuels and particularly diesel and jet fuels. In addition, the fatty alcohols or derivatives produced there from can be combined with other fuels or fuel additives to produce fuels having desired properties. Such other fuels may include traditional fuels, such as alcohols and petroleum based fuels. Fuel additives may include but are not limited to cloud point lowering additives and surfactants. In some embodiments, the fuel composition comprising a fatty alcohol produced according to the invention and derivatives thereof having C8 to C24 will include at least about 5%, 10%, 15%, 20%, 25%, 30%, 35%, 40%, 45%, 50%, 55%, 60%, 65%, 70%, 75%, and 80% of the fatty alcohol or derivative thereof. In some embodiments, the percent of fatty alcohols or derivatives thereof will be between 5% and 50%. In some embodiments the percent will be greater than 5% but less than 60%.

In some embodiments, the term “biofuel” composition is used to distinguish a fuel composition comprising a fatty alcohol or derivative thereof made by the biological process as disclosed herein which includes production and/or secretion of a fatty alcohol from a recombinant microorganism which is grown on a carbon source from renewable feedstock as opposed to a fatty alcohol or derivative thereof made from a petroleum based carbon source.

### 7. EXAMPLES

Various features and embodiments of the disclosure are provided in the following representative examples, which are intended to be illustrative and not limiting.

### Example 1

### Gene Acquisition

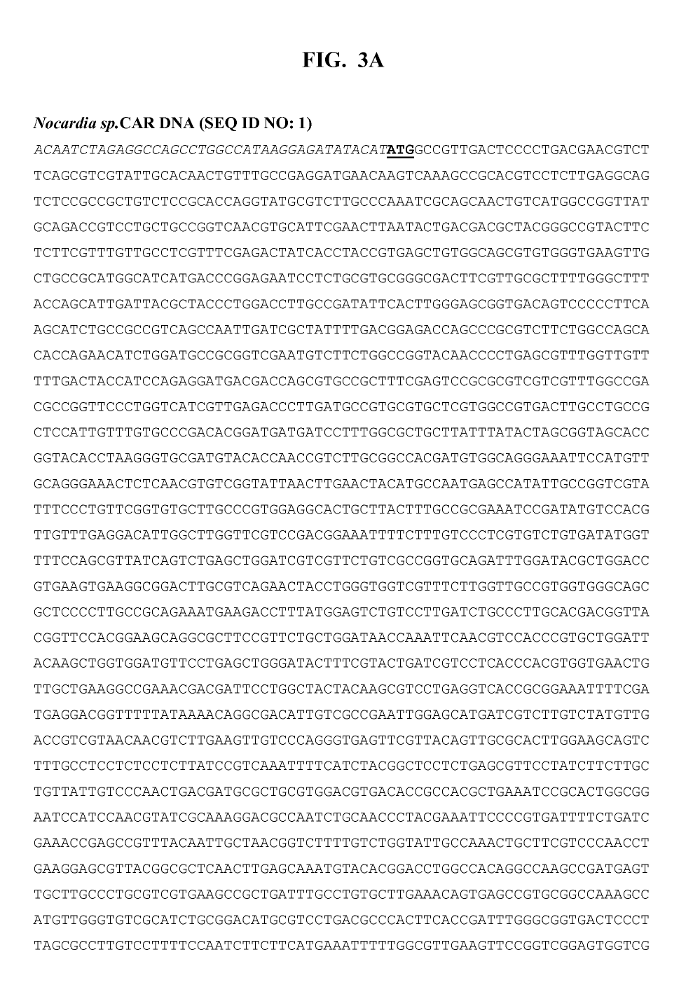

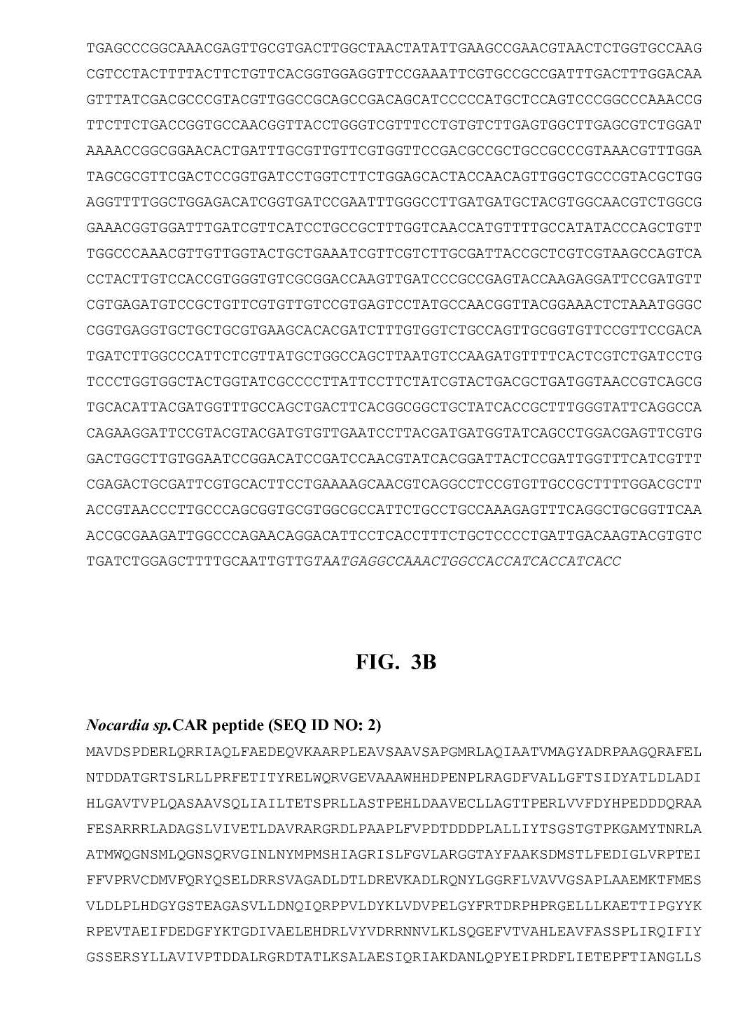

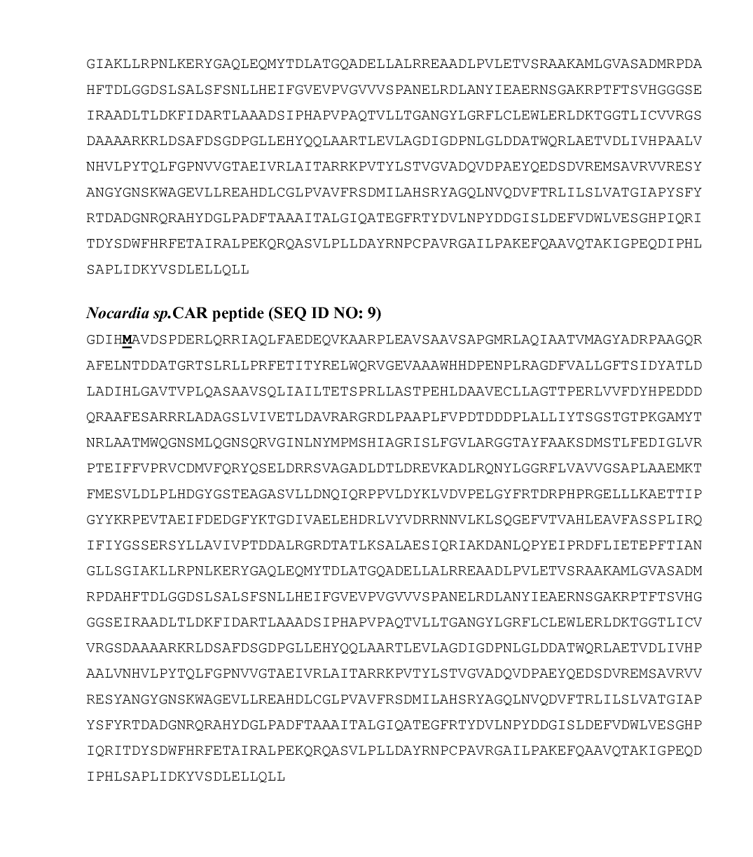

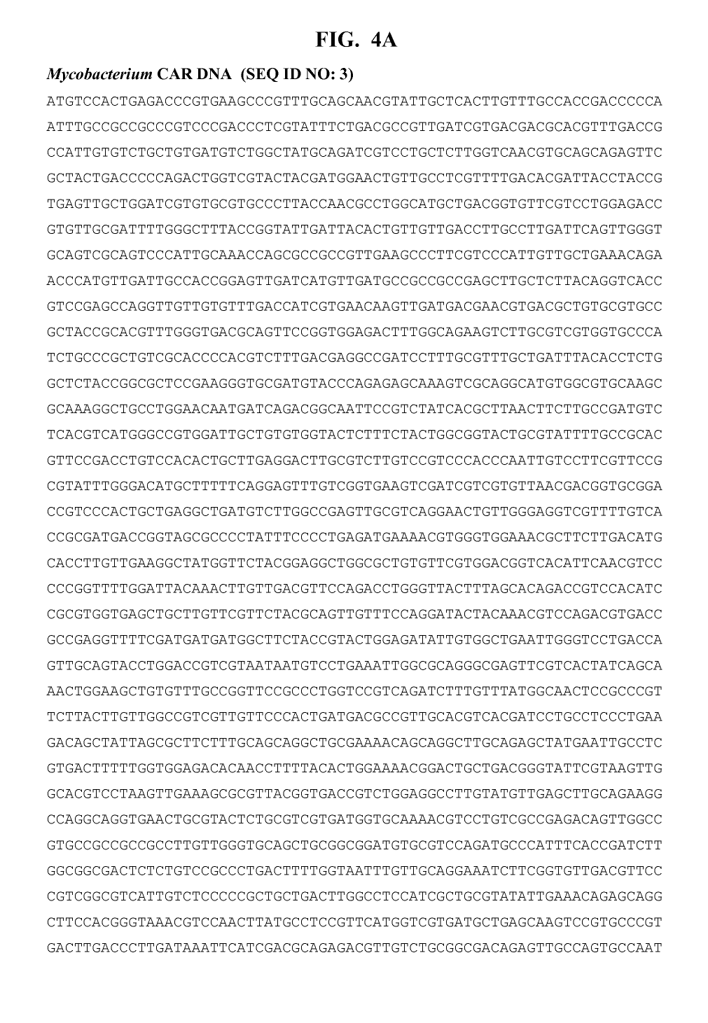

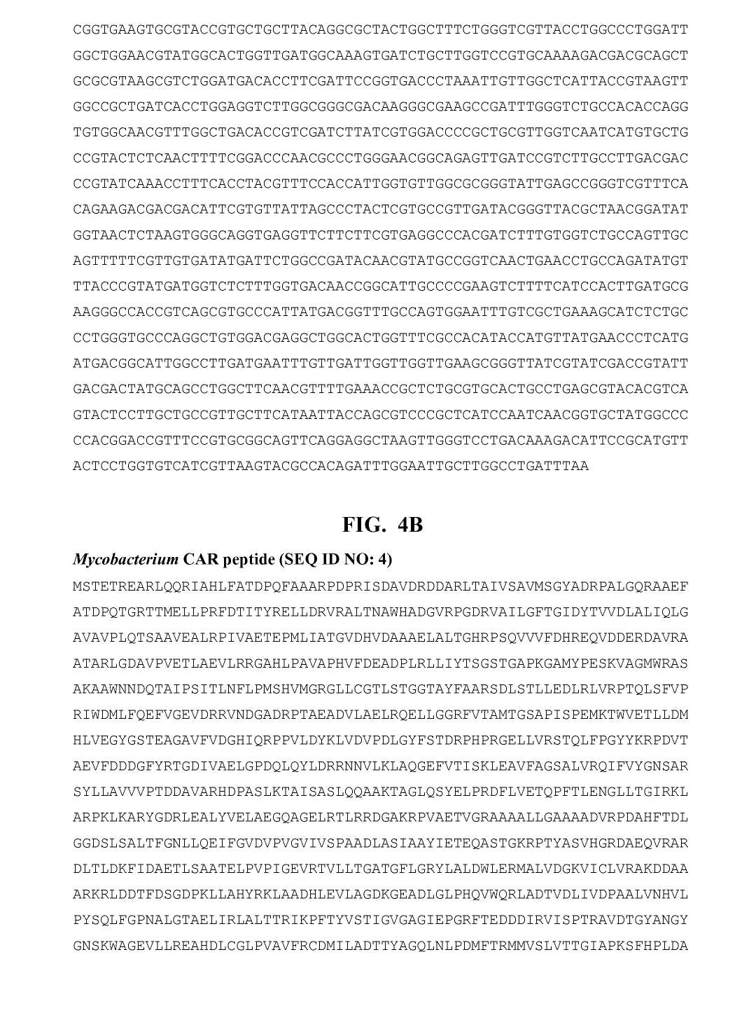

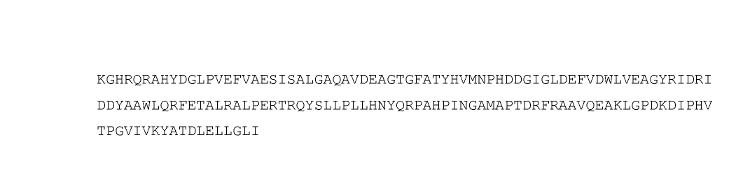

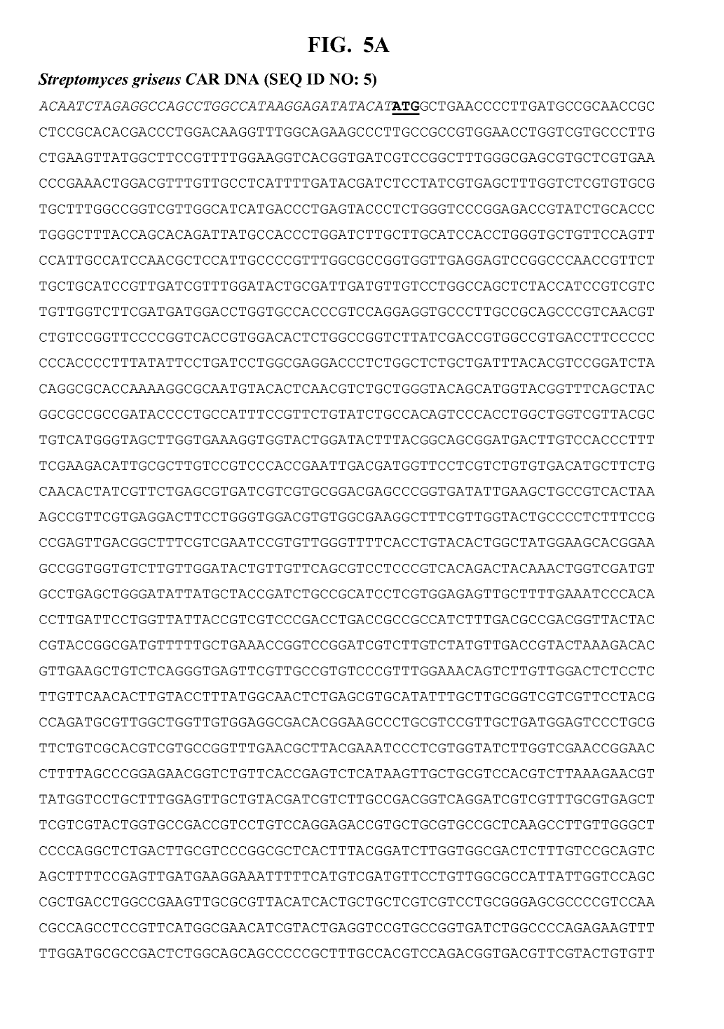

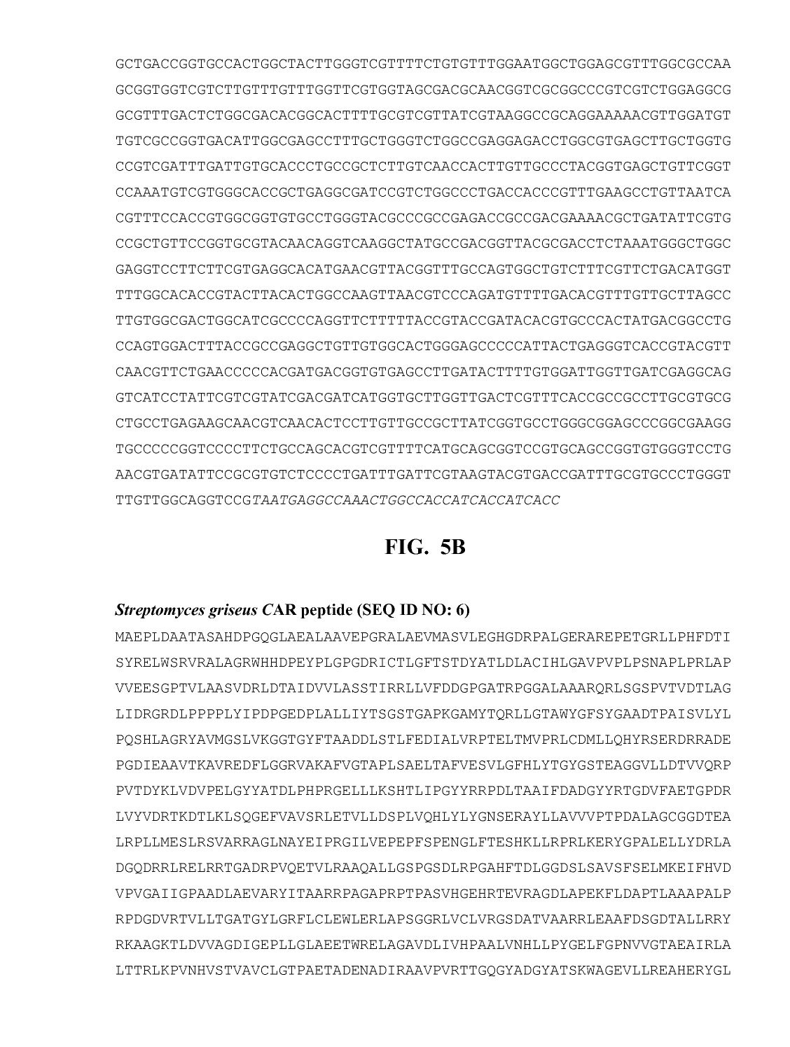

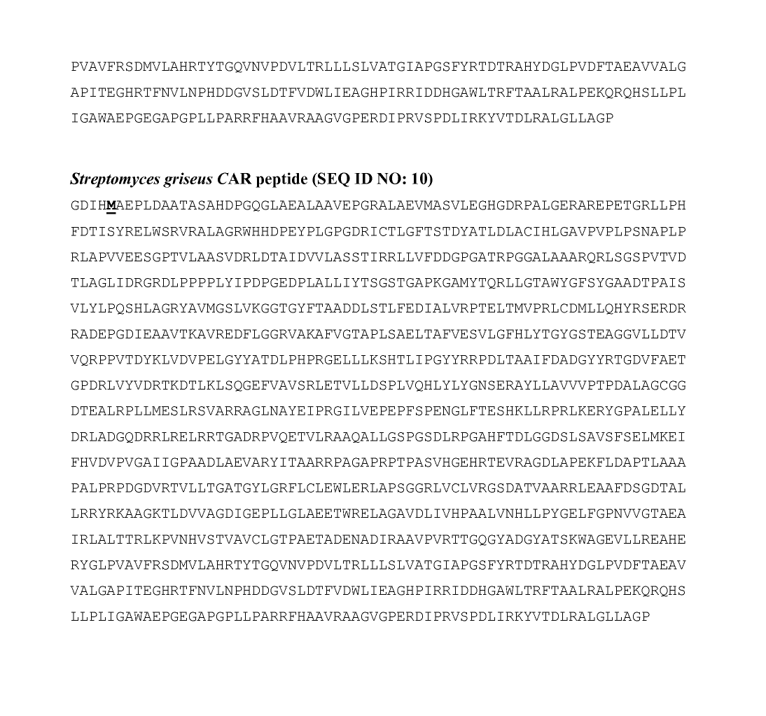

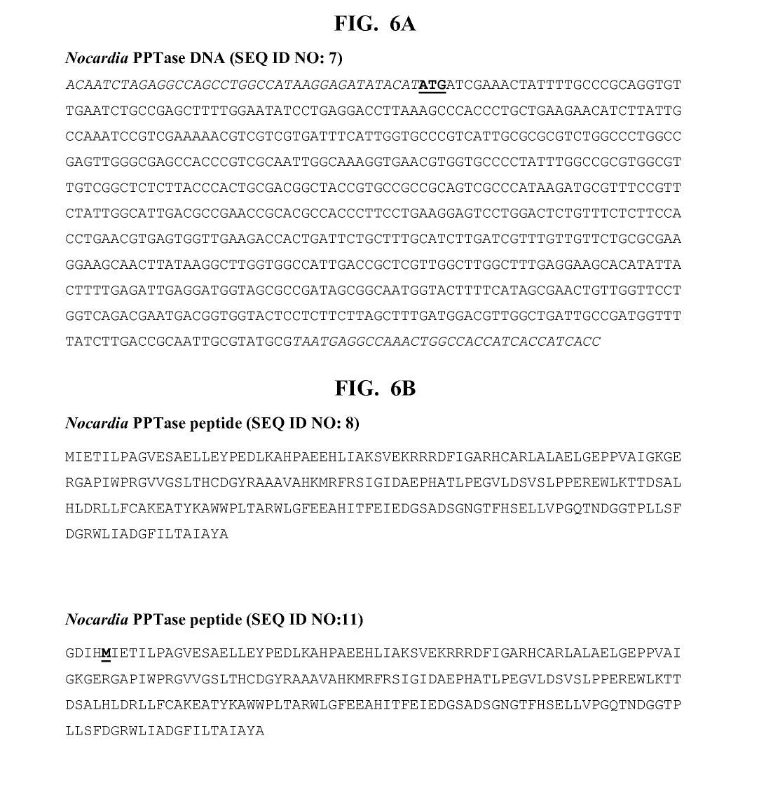

Wild-type Nocardia NRRL5646, Mycobacteria sp. JLS, and Streptomyces griseus carboxylic acid reductases (CARs) and Nocardia phosphopantetheine transferase (PPTase) genes were designed for expression in E. coli, S. cerevisiae, and Y. lipolytica based on the reported amino acid sequences (Nocardia CAR: Appl Environ Microbiol (2004) v 70 p1874, S. griseus CAR: J. Bacteriol. 190 (11), 4050-4060 (2008), Mycobacteria CAR: GenBank accession number YP 001070587, Nocardia PPTase: Biol. Chem. 282 (1), 478-485 (2007). Codon optimization was performed using an algorithm as described in Example 1 of WO2008042876 incorporated herein by reference. The genes were synthesized by Genscript (Piscataway, N.J.) with flanking restriction sites for cloning into E. coli vector pCK 110900 described in US Pat. Appln. Pub. 2006 0195947. Nucleotide sequences for SfiI sites were added to the 5′ end and 3′ end of the gene as well as the t7 g10 RBS in front of the ATG start codon. The genes were provided in the vector pUC57 by Genscript (Piscataway, N.J.) and the sequences verified by DNA sequencing. The sequences of the codon optimized genes correspond to SEQ ID NO: 1, SEQ ID NO: 3, SEQ ID NO: 5 and SEQ ID NO: 7. The corresponding expressed polypeptide sequences are designated SEQ ID NOs: 2 and 9; SEQ ID NO: 4; SEQ ID NOs: 6 and 10; and SEQ ID NOs: 8 and 11. Reference is made to FIGS. 3, 4, 5, and 6.

### Example 2

### Expression and Activity of CARs and PPTase in E. Coli

### (a) Construction of Vectors to Express CARs and PPTase in E. Coli.

The genes were cloned into the vector pCK11900-I (depicted as FIG. 3 in US Pat. Appln. Pub. 2006 0195947) under the control of a lac promoter using the Sfi I restriction sites. The PPTase gene as well as the t7 g10 RBS was added upstream of the ATG start codon for each of the CAR genes by restriction free cloning (J Biochemical Biophysical Methods, 2006, 67-74. The expression vector also contained the P15a origin of replication and the chloramphenicol resistance gene. The resulting plasmids were introduced into E. coli BW25113 (ΔfadE) (Nature 418(6896):387-9, (2002)) by routine transformation methods.

### (b) In Vivo Activity of Cars in Recombinant E. Coli.

Recombinant E. coli strains comprising a plasmid containing a heterologous gene encoding either the Nocardia NRRL5646, the Mycobacteria sp. JLS, or the Streptomyces griseus carboxylic acid reductase, were grown in Luria Bertani Broth (LB) medium supplemented with 1% glucose and 30 μg/mL chloramphenicol (CAM), for approximately 16-18 hours (overnight) at 30° C., in a shaker incubator at 200 rpm. A 5% inoculum was used to initiate fresh 50 mL Luria Bertani Broth (LB) culture supplemented with 30 μg/mLCAM. The culture was incubated for 2.5 hours 30° C., 200 rpm to an OD₆₀₀ of about 0.6 to about 0.8, at which point expression of the heterologous carboxylic acid was induced with isopropyl-β-D-thiogalactoside (IPTG) (1 mM final concentration). Incubation was continued for about 16 hours (overnight) under the same conditions. Cells were collected by centrifugation for 10 minutes at 6000 rpm in F15B-8×50C rotor. Aliquots of 40 OD₆₀₀ units of each culture were centrifuged and the cell pellets were resuspended in 0.5 mL of 6.7% Na₂SO₄ and then extracted with isopropanol:hexane (0.8:1.2) for 20 minutes. The extract was centrifuged and a 400 μL, sample taken of the top organic layer. The solvent in the sample was evaporated under a nitrogen stream and the residue derivatized with 100 μL N,O-Bis(trimethylsilyl)trifluoroacetamide) (BSTFA) at 37° C. for 1 hour, and then diluted with 100 μL of heptanes before analysis by GC-FID and GC-MS. In addition, 0.5 mL of the culture medium (after removal of cells by centrifugation) was combined with 0.5 mL methanol:chloroform (1:1) and extracted for 20 minutes. The lower organic phase was collected, solvent evaporated and the residue derivatized with BSTFA as above. A 1 μL sample was analyzed by GC-MS or GC-FID with the split ratio 1:10. GC parameters: initial oven temperature 80° C. and held at 80° C. for 3 minutes. The oven temperature was increased to 200° C. at a rate of 50° C./minute followed by rate of 10° C./minute to 270° C., and then 20° C./minute to 300° C., and then held at 300° C. for five minutes. Under the conditions tested, expression of both the Nocardia NRRL5646 and Mycobacteria sp. JLS carboxylic acid reductase in E. coli resulted in the intracellular production of long chain fatty alcohols (see Table 2). In both cases PPTase improved the activity of the CAR enzyme from 1 to 2 times. Secreted fatty alcohols were not detected. Identification of individual fatty alcohol was done by comparison to commercial standards (Sigma Chemical Company, 6050 Spruce St. Louis, Mo. 63103).

TABLE 2
Fatty alcohol profile exhibited by recombinant E. coli host cells
/over-expressing heterologous CAR genes.

| | | | | | | | Estimated |
|---|---|---|---|---|---|---|---|
| | Cellular fatty alcohol composition^{a} | | | | | | productivity^{b} |
| Enzyme | C12:0 | C14:0 | C16:1 | C16:0 | C18:1 | C18:0 | (μg/OD600) |
| Nocardia CAR | <10 | <10 | 20-40 | 20-40 | 20-40 | ND | 0.1-0.5 |
| Mycobacterium CAR | <10 | >40 | 20-40 | 10-20 | ND | ND | <0.1 |

^{a}The relative amounts of each fatty alcohol component are expressed as a % of the total fatty alcohols detected using TMS derivative via GC/FID or GC/MS. Endogenous fatty alcohols include: C12:0 (1-dodecanol), no C12:1 (1-dodecenol) was detected, C14:0 (1-tetradecanol), no C14:1 (1-tetradecenol) was detected, C16:1 (cis Δ⁹-1-hexadecenol), C16:0 (1-hexahecanol), C18:1 (cis Δ¹¹-1-octadecenol), 18:0 (1-octadecanol).
ND: not detected.
^{b}Enzyme productivity was estimated using external standard (1 OD600 unit corresponds to approximately 0.3 mg of cells).

### (c) In Vitro Activity of Cars in Recombinant E. Coli.

Preparation of cell pellets containing CARs and PPTase in 96-well plates:

The recombinant E. coli strains comprising a plasmid containing a heterologous gene encoding either the Nocardia NRRL5646, the Mycobacteria sp. JLS, or the Streptomyces griseus carboxylic acid reductase and the Nocardia PPTase, were grown in a 96-well shallow plate containing 180 μL Luria Bertani Broth (LB), supplemented with 1% glucose and 30 μg/mL chloramphenicol (CAM), for approximately 16 hours (overnight) at 30° C., 200 rpm at 85% humidity. A 20 μL of each seed culture was transferred into 390 μL Terrific Broth (TB) supplemented with 30 μg/mL CAM, in an individual well of a 96-deep well plate. The latter plate was incubated for 2.5 hours 30° C., 200 rpm at 85% humidity to an OD₆₀₀ of about 0.6 to about 0.8, at which point expression of the heterologous carboxylic acid was induced with isopropyl-β-D-thiogalactoside (IPTG) (1 mM final concentration). Incubation was continued for about 16 hours (overnight) under the same conditions. Cells were collected by centrifugation for 10 minutes at 4000 rpm.

Preparation of crude lysate of CAR enzymes and PPTase in 96-well plates:

To each well of the 96-deep well plate containing the pelleted cells prepared above, 0.4 mL of lysis buffer (100 mM KH2PO4, pH 7.5, 1 mg/mL lysozyme, 0.5 mg/mL polymixin B sulfate (PMBS) was added. Cells were lysed for 2 h at room temperature with shaking on a bench-top shaker. Each plate was then centrifuged for 10 minutes at 4000 rpm at 4° C. The clear supernatant recovered after the centrifugation was recovered and used for the biochemical assays.

In vitro activity of CARs in 96-well plates using spectroscopic method: An aliquot of the supernatant obtained above was added to the assay mixture comprising 100 mM phosphate buffer (pH 7.5), 0.2 mg/mL NADPH, 1 mM ATP, 1 mM CoA and 5 mM substrate (e.g., benzoic acid, octanoic acid, and decanoic acid). The reaction was monitored by measuring the decrease of fluorescent emission of NADPH at 440 nm as a function of time. The results were plotted as relative fluorescent units (RFU) of NADPH versus time. The slope of the plot (RFU/min) was used to determine the rate of reaction.

In vitro activity of CARs in 96-well plates using GC method:

An aliquot (20 μL) of the CAR/PPTase supernatant obtained above was added to the assay mixture comprising 100 mM phosphate buffer (pH 7.5), 0.2 mg/mL NADPH, 2 mM ATP, 1 mM CoA, 4 mM hexadecanoic acid and 3% isopropyl alcohol (IPA). An engineered ketoreductase enzyme (2 mg/mL) (SEQ ID NO. 77 in WO2008103248A1) was added to regenerate NADPH in the reaction by converting IPA to acetone. After overnight incubation at room temperature on a bench top shaker, the reaction mixture was extracted by 600 μL MTBE containing methyl hexadecanoate as an internal standard. A 1 μL sample was analyzed by GC-MS or GC-FID with conditions similar to those as described above (example 2b). Under the conditions tested approximately 50% conversion of substrate was detected by both Mycobacteria and Nocardia CARs. The data obtained indicated that both the enzymes (coupled with an apparent background E. coli alcohol dehydrogenase/ketoreductase activity) converted hexadecanoic acid to hexadecanol. Under the conditions tested, the Streptomyces griseus CAR did not display significant activity.

### (d) In Vitro Screening of Mycobacterium CAR Variants in Recombinant E. Coli.

Random and targeted mutagenesis of Mycobacterium CAR was used to generate variants that were screened (growth, lysis, and assay) as described in examples 2C I, II and IV. A number of variants with 0.7 to 3.4-fold activity relative to wt Mycobacterium CAR (SEQ ID NO: 4) were identified (Table 3). Combinations of these mutations is expected to further improve the relative activity compared to wt Mycobacterium CAR.

TABLE 3
Amino acid substitutions and relative activity of Mycobacterium
CAR variants. The variants are aligned and compared to the
CAR sequence of SEQ ID NO: 4.

| Sequence changes (Compare to WT | Relative activity (compare to WT |
|---|---|
| Mycobacterium CAR) | Mycobacterium CAR) |
| A271W | 0.8 |
| A275F | 1.8 |
| D701G | 0.7 |
| E626G | 1.9 |
| K274G | 1.1 |
| K274L; A369T; L380Y | 2.1 |
| K274L; V358H; E845A | 2.5 |
| K274M; T282K | 2.3 |
| K274N | 1.6 |
| K274Q; T282Y | 1.2 |
| K274S; A715T | 0.9 |
| K274V | 1.8 |
| K274W; L380F | 3.4 |
| K274W; L380G; A477T | 2.6 |
| K274W; T282E; L380V | 2.5 |
| K274W; T282Q | 3.3 |
| K274W; V358R | 3.4 |
| P467S | 1.2 |
| Q584R | 1.0 |
| R270W | 1.1 |
| R43C; K274I | 2.9 |

### Example 3

### Expression and Activity of CARs in S. cerevisiae

(a) Construction of Vectors to Express CARs and PPTase in S. cerevisiae.

The Nocardia PPTase gene was PCR amplified and cloned downstream of the GAL10 promoter using NotI and SpeI sites into vector pESC-LEU (Stratagene, La Jolla, Calif.) to construct pCENO314. The CAR genes were PCR amplified and cloned using BamHI and SalI sites into vector pCENO314 under the control of the GAL1 promoter. These vectors contain a 2 micron yeast origin and LEU2 gene for selection in S. cerevisiae YPH499 (Stratagene, La Jolla, Calif.).

(b) In Vivo Activity of CARs in Recombinant S. cerevisiae Host Strains Using Shake Flasks.

The recombinant S. cerevisiae strains comprising a plasmid containing a heterologous gene encoding either the Nocardia NRRL5646, the Mycobacteria sp. JLS, or the Streptomyces griseus carboxylic acid reductase gene, were inoculated in 5 ml of YNB (Yeast Nitrogen Base)-Leu containing 2% glucose (SD media) and grown at 30° C. for overnight (OD ˜3). Approximately 2.5 ml were subcultured into 50 ml (20× dilution, OD ˜0.15) of SD media and grown at 30° C. for 8 hours to OD ˜1. Cell cultures were centrifuged at ˜3000-4000 rpm (F15B-8×50C rotor) for 10 minutes, the supernatant was discarded. The residual medium was removed with the pipette or the cells were washed with SG medium (YNB-Leu containing 2% galactose). The pellets were resuspended in 250 mL SG media (5× dilution, starting culture ˜OD 0.2,) and grown overnight at 30° C. before harvesting.

For extraction and identification of intra-cellular fatty alcohols, 30-50OD₆₀₀ units of cells were centrifuged and pellets were washed with 20 ml of 50 mM Tris-HCl pH7.5. Cells were resuspended in 0.5 ml of 6.7% Na₂SO₄, and transferred into 2 ml tubes. 0.4 ml of isopropanol and 0.6 ml of hexane were added and the mixture was vortexed for ˜30 minutes and centrifuged for 2 minutes at 14,000 rpm using a bench top centrifuge (eppendrof F45-25-11). The upper organic phase was collected and evaporated under a nitrogen stream. The remaining residue was derivatized with 100 μL BSTFA at 37-60 C for 1 hour, left at room temperature for another 3 to 12 hours and diluted with 100 μL heptane before analysis by GC-FID or GC-MS.

For extraction and identification of extra-cellular fatty alcohols, 1 ml of 1:1 (vol:vol) chloroform:methanol was added to 0.5 ml of culture supernatant, vortexed for ˜30 min, and centrifuged for 2 minutes at 14,000 rpm using a bench top centrifuge (eppendrof F45-25-11). The upper phase was discarded and the ˜1 ml of the lower phase was transferred to a 2 ml autosampler vial. The extracts were dried under a nitrogen stream and the residue was derivatized with 100 ul BSTFA at 37-60° C. for 1 hour and 3 to 12 hours at room temperature. The mixture was diluted with 100 μl heptane before analysis by GC-FID or GC-MS. GC conditions were similar to those provided in example 2b. Under the conditions tested, expression of both the Nocardia NRRL5646, the Mycobacteria sp. JLS carboxylic acid reductase in S. Cerevisiae YPH499 resulted in the intracellular production of long chain fatty alcohols (see Table 4). Secreted fatty alcohols were not detected.

TABLE 4
Fatty Alcohol Profile Exhibited by Recombinant S. cerevisiae Cells Over-
Expressing the Heterologous Enzyme Genes

| | | | | | | | Estimated |
|---|---|---|---|---|---|---|---|
| | Cellular fatty alcohol composition^{a} | | | | | | productivity^{b} |
| Enzyme | C12:0 | C14:0 | C16:0 | C16:1 | C18:0 | C18:0 | (mg/g DCW) |
| Nocardia CAR | 12 | 10 | 33 | 38 | trace | 6 | 0.3 |
| Mycobacterium CAR | 10 | 10 | 38 | 27 | 7 | 8 | 0.4 |

^{a}The relative amounts of each fatty alcohol component are expressed as a percent of the total fatty alcohols detected using TMS derivative via GC/FID or GC/MS. Endogenous fatty alcohols include C12:0 (1-dodecanol), C14:0 (1-tetradecanol), C16:0 (1-hexadecanol), C18:1 (cis Δ⁹-1-octadecenol), and 18:0 (1-octadecanol).
DCW = dry cell weight.

### Example 4

### Expression and Activity of CAR Enzymes in Yarrowia lipolytica

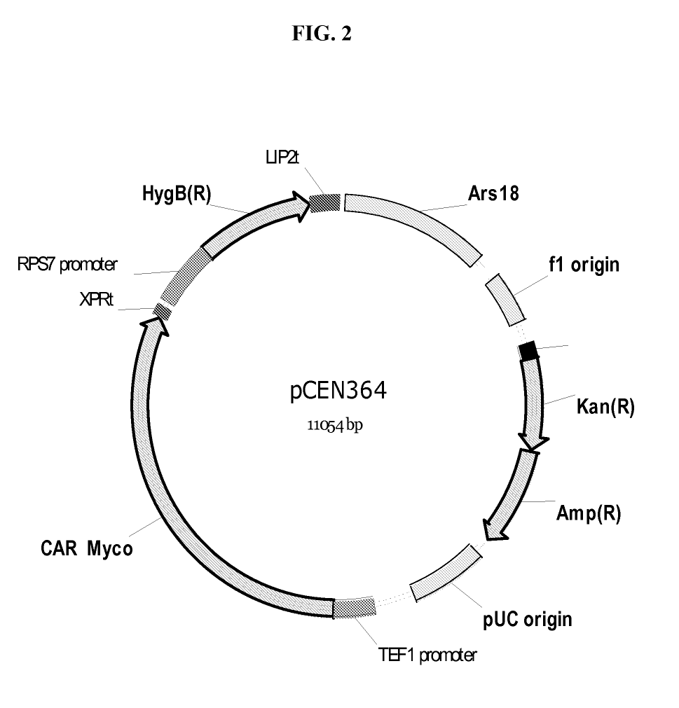

An autonomous replicating plasmid for expression of genes in Y. lipolytica was engineered with two antibiotic selection marker cassettes for resistance to hygromycin and phleomycin (HygB(R) or Ble(R), respectively) (plasmid pCEN351, FIG. 1). Expression of each cassette is independently regulated by a strong, constitutive promoter isolated from Y. lipolytica: pTEF1 for Ble(R) expression and pRPS7 for HygB(R) expression. Plasmid pCEN351 was used to assemble Y. lipolytica expression plasmids. Using “restriction free cloning” methodology, the Mycobacterium CAR in Y. lipolytica gene was inserted into pCEN351 to provided plasmid pCEN364 (FIG. 2). In pCEN364, heterologous gene expression is under control of the constitutive TEF 1 promoter. The HygB^{R} gene allows for selection in media containing hygromycin. Ars18 is an autonomous replicating sequence isolated from Y. lipolytica genomic DNA. The resulting plasmid (pCEN364) was transformed by standard procedures into Y. lipolytica 1345 which was obtained from the German Resource Centre for Biological Material (DSMZ).

(a) In Vivo of CAR Activity in Recombinant Y. lipolytica.

The recombinant Y. lipolytica strains comprising plasmid containing a heterologous gene encoding either the Nocardia NRRL5646, the Mycobacteria sp. JLS, or the Streptomyces griseus carboxylic acid reductase, were inoculated in 200 mL YPD media containing 500 μg/mL hygromycin. The cultures were grown at 30° C. to an OD600 of 4-7. Cells were then harvested by centrifugation and washed with 20 ml of 50 mM Tris-HCl pH7.5. Extraction and identification of intra-cellular fatty alcohols were performed as described in Example 3b. Under the conditions tested trace amount of 1-hexadecanol and 1-octadecanol were detected by Nocardia CAR. Secreted fatty alcohols were not detected.

(b) In Vitro of CAR Activity in Recombinant Y. lipolytica.

The recombinant Y. lipolytica strains comprising a plasmid containing a heterologous gene encoding either the Nocardia NRRL5646, the Mycobacteria sp. JLS, or the Streptomyces griseus carboxylic acid reductase, were inoculated in 200 mL YPD media containing 500 μg/mL hygromycin. The cultures were grown at 30° C. to an OD600 of 4-7. Cells were then harvested by centrifugation, washed and stored at −80° C. For lysis, cell pellets were resuspended in 15 mL of 100 mM sodium phosphate pH 7.0. The cell suspension was supplemented with protease inhibitor tablets (Calbiochem #539137), then placed into a stainless steel bead beater (15 mL capacity) loaded with glass beads. The bead beater was submerged into an ice bath, and cells were lysed using ten cycles of bead beating for 30 seconds followed by cooling for 90 seconds. The lysate was centrifuged at 15,000 rpm in JA25.50 rotor for 20 minutes. The total protein concentration was estimated to be 9-16 mg/ml. E. coli lysate containing the Nocardia PPTase was prepared as described in Examples 1 and 2. An aliquot (4-6 mL) of the Y. lipolytica CAR lysate obtained above was pre-incubated for ˜1 hr with 1.5 mL of the E. coli Nocardia PPTase lysate. 110 μL of this mixture was then added to the assay mixture comprising 100 mM phosphate buffer (pH 7.5), 0.2 mg/mL NADPH, 2 mM ATP, 1 mM CoA, 1 mM hexadecanoic acid and 3% IPA and 2 mg/mL ketoreductase (SEQ ID NO. 77 in WO2008103248A1) to regenerate NADPH. After 4 hrs (for Nocardia CAR) and 19 hrs (for Mycobacterium CAR) incubation at room temperature on bench top shaker, the reaction mixture was extracted by 600 μL MTBE containing methyl hexadecanoate as internal standard. A 1 μL sample was analyzed by GC-MS or GC-FID with the conditions described above. Under the conditions tested approximately 90% conversion of hexadanoic acid to 1-hexadecanol was detected by both Mycobacteriaum and Nocardia CARs. PPTase was observed to improve CAR activity for both Nocardia CAR and Mycobacterium CAR by 3-5 times and by 9-20 times respectively activity The inventors believe the conversion of the hexadecanyl aldehyde to the corresponding alcohol occurs by endogenous ketoreductase activity in Y. lipolytica.

All publications, patents, patent applications and other documents cited in this application are hereby incorporated by reference in their entireties for all purposes to the same extent as if each individual publication, patent, patent application or other document were individually indicated to be incorporated by reference for all purposes.

What is claimed is:

1. A process for the biologically-derived production of fatty alcohols in yeast comprising:
a) culturing a recombinant yeast cell, which comprises a polynucleotide encoding a heterologous carboxylic acid reductase (CAR) under suitable culture conditions to allow expression of said CAR and production of the fatty alcohols, and
b) recovering the fatty alcohols produced by the recombinant yeast cell.

2. The process according to claim 1, wherein the yeast is a Yarrowia strain, Candida strain or Saccharomyces strain.

3. The process according to claim 1, wherein the recombinant yeast are capable of producing fatty alcohols comprising C10 to C20 carbons in length.

4. The process according to claim 1, wherein the amount of fatty alcohol produced is at least 2.0 mg/L of culture media.

5. The process according to claim 1, wherein the heterologous CAR has at least 90% sequence identity to SEQ ID NOs: 2, 4, 6, 9 or 10.

6. The process according to claim 1, wherein the recombinant yeast further comprises a gene encoding a heterologous phosphopantetheinyl transferase capable of attaching a phosphopantetheine moiety to the CAR.

7. The process according to claim 1, wherein the polynucleotide coding for the CAR comprises a sequence having at least 90% sequence identity to SEQ ID NO: 1, 3 or 5.

8. The process according to claim 1, wherein the recombinant yeast cell further comprises a polynucleotide encoding a heterologous alcohol dehydrogenase (ADH).

9. A biologically-derived fatty alcohol composition produced by the process of claim 1.

10. A process for the biologically-derived production of fatty alcohols comprising:
a) culturing a recombinant microorganism, which comprises
i) a polynucleotide coding for a heterologous carboxylic acid reductase (CAR) comprising an amino acid sequence having at least 90% sequence identity to SEQ ID NOs: 2, 4, or 6, and
ii) a polynucleotide coding for a heterologous phosphopantetheinyl transferase (PPTase) having at least 80% sequence identity to SEQ ID NO: 8, wherein said PPTase is capable of attaching a phosphopantetheine moiety to the CAR under suitable culture conditions to allow the expression of the CAR and PPTase and production of the fatty alcohols, and
b) recovering the produced fatty alcohol.

11. The process according to claim 10, wherein the recombinant microorganism is a bacterial strain, a yeast strain, a filamentous fungal strain or an algal strain.

12. The process according to claim 10, wherein the CAR and the PPTase are derived from the same organism.

13. A recombinant microorganism comprising a nucleic acid sequence encoding a heterologous carboxylic acid reductase, wherein the recombinant microorganism is capable of producing at least 2 mg/L of fatty alcohols having C8 to C24 carbons in length.

14. The recombinant microorganism of claim 13, wherein the carboxylic acid reductase is selected from the group consisting of a Mycobacterium carboxylic acid reductase, a Nocardia carboxylic acid reductase, and a Streptomyces griseus carboxylic acid reductase.

15. The recombinant microorganism of claim 14, wherein the recombinant microorganism is a bacterial strain, a filamentous fungal strain, a yeast strain or an algal strain.

16. The recombinant microorganism of claim 13, wherein the recombinant microorganism comprises a gene encoding a phosphopantetheinyl transferase polypeptide capable of attaching a phosphopantetheine moiety to the carboxylic acid reductase.

17. The recombinant microorganism of claim 13, wherein the amount of fatty alcohol produced is at least 5 mg/L.

18. A process for the biologically-derived production of fatty alcohols comprising:
a) culturing the recombinant microbial host cell according to claim 13 in an aqueous nutrient medium comprising an assimilable source of carbon under suitable culture conditions for a sufficient period of time to allow the production the fatty alcohols, and
b) isolating the produced fatty alcohols.

19. The process according to claim 18, wherein the culturing is carried out at a temperature within the range of from about 10° C. to about 80° C. and for period of from about 8 hours to about 240 hours.

20. The process according to claim 18, wherein the amount of biologically produced fatty alcohol is in the range of 2 mg/L to 200 g/L.

21. The process according to claim 18, wherein the production of fatty alcohols having C10 to C20 carbons in length comprises at least 80% of the total isolated fatty alcohols.

22. A biologically-derived fatty alcohol composition comprising the fatty alcohols or derivatives of said fatty alcohols, wherein the fatty alcohols are produced according to the process of claim 18.

23. The fatty alcohol composition of claim 22 produced by a recombinant E. coli strain.

24. The process of claim 18, further comprising reducing the fatty alcohols to corresponding alkanes.

25. A method of catalytically reducing a fatty acid substrate to a corresponding C8 -C24 carbon containing fatty aldehyde comprising a) mixing an effective amount of an isolated carboxylic acid reductase, with a fatty acid substrate and cofactors selected from the group of ATP and NADPH and b) incubating the mixture for a period of time and under conditions suitable to achieve reduction of the substrate to the corresponding fatty aldehyde.

26. The method according to claim 25 further comprising reducing the fatty aldehyde to a fatty alcohol.

27. The method according to claim 26, wherein the carboxylic acid reductase is selected from the group of a Mycobacterium sp. JLS carboxylic acid reductase, a Nocardia sp. carboxylic acid reductase, and a Streptomyces griseus carboxylic acid reductase.

28. An isolated carboxylic acid reductase (CAR) variant comprising at least 90% sequence identity to SEQ ID NO: 4 and an amino acid substitution at one or more of the following positions R270, A271, K274, A275, P467, Q584, E626, and/or D701 when aligned with SEQ ID NO: 4.
